(19) United States
(12) Patent Application Publication
(10) Pub. No.: US 2022/0331442 A1
(43) Pub. Date: Oct. 20, 2022

(54) ANTIBODY-BIOMOLECULE CONJUGATES LINKED THROUGH MULTIFUNCTIONAL MACROMOLECULE AND USES THEREOF

(71) Applicant: LEVIM BIOTECH LLP, Tamilnadu, Chennai (IN)

(72) Inventors: Raman SRIKAR, Egattur, Chennai (IN); Vimal JATIN, T. Nagar, Chennai (IN); K. SREEDURGALAKSHMI, Mandaveli, Chennai (IN)

(21) Appl. No.: 16/981,095

(22) PCT Filed: Mar. 15, 2019
(86) PCT No.: PCT/IB19/52124

§ 371 Date: Sep. 15, 2020

(30) Foreign Application Priority Data
Mar. 15, 2018 (IN) 201741032754

(51) Int. Cl. A61K 47/64; A61K 47/68; C12N 15/113; A61P 35/00

(52) U.S. Cl. CPC A61K 47/6455; A61K 47/6849; A61K 47/6807; A61K 47/6857; C12N 15/1135; A61P 35/00; C12N 2310/14; C12N 2310/3513; C12N 2320/32

(57) ABSTRACT

The present invention relates to a conjugate for delivery of biomolecules using a cationic macromolecule as a linker between the biomolecule and biomarker targeting moiety, and the process of making the conjugate.

### FIELD OF THE INVENTION

The present disclosure provides a novel antibody-biomolecule conjugate delivery system for stable delivery of biomolecule for therapeutic and/or diagnostic application, the method of synthesis and uses thereof.

### BACKGROUND OF THE INVENTION

Short Interfering RNA (siRNA) is a promising therapeutic tool for genetic diseases such as cancer, wherein several types of carcinomas are either undruggable or develop mutation leading to drug resistance [Morgillo, F., et al., Mechanisms of resistance to EGFR-targeted drugs: lung cancer. ESMO Open, 2016. 1(3): p. e000060; Stewart, E. L., et al., Known and putative mechanisms of resistance to EGFR targeted therapies in NSCLC patients with EGFR mutations—a review. Transl Lung Cancer Res, 2015. 4(1): p. 67-81; de Castro Carpeno, J. and C. Belda-Iniesta, KRAS mutant NSCLC, a new opportunity for the synthetic lethality therapeutic approach. Transl Lung Cancer Res, 2013. 2(2): p. 142-51]. Knocking down the oncogene of interest circumvents the drugability issues enabling effective personalized therapies [Whitehead, K. A., R. Langer, and D. G. Anderson, Knocking down barriers: advances in siRNA delivery. Nat Rev Drug Discov, 2009. 8(2): p. 129-38]. siRNA is known for effective knockdowns of cytoplasmic genes with high specificity leading to cellular apoptosis or drug sensitization. However, their clinical application is restricted due to its poor in-vivo stability [Hickerson, R. P., et al., Stability study of unmodified siRNA and relevance to clinical use. Oligonucleotides, 2008. 18(4): p. 345-54]. siRNAs are extremely sensitive to enzymes present in serum and undergo rapid degradation. The half-life of siRNA in serum is known to be only several minutes inhibiting sufficient accumulation of the compound within target site for effective therapeutic action. Also, siRNAs need to localize within cytoplasm of cells for effective mRNA knockdown. Localization within cytoplasm or cytoplasmic translocation requires endosomal escape after cellular internalization which is generally achieved if the molecule of interest is cationic or has charge reversal properties [Guo, S., et al., Enhanced gene delivery and siRNA silencing by gold nanoparticles coated with charge-reversal polyelectrolyte. ACS Nano, 2010. 4(9): p. 5505-11]. To overcome these challenges, several delivery systems have been reported that can protect the siRNA from external degradation factors and effect stable delivery within the cytoplasm of cells [Sarisozen, C, Salzano G, and Torchilin V. P., Recent advances in siRNA delivery. Biomol Concepts, 2015. 6(5-6): p. 321-41].

Most prominent systems reported for the delivery of siRNA is based on nanoparticle (NP) platform [Williford, J. M., et al., Recent advances in nanoparticle-mediated siRNA delivery. Annu Rev Biomed Eng, 2014. 16: p. 347-70.]. Metallic, polymeric and liposomal based NP systems has been extensively investigated for the delivery of siRNA. In the case of metallic nanoparticles, the most commonly used material for designing the delivery system is gold nanoparticles (AuNP). AuNP delivery system have emerged as a lead molecule for proof of concept studies. Two adopted methods for AuNP mediated biomolecule delivery includes chemical conjugation of siRNA to AuNP, for instance via thiol-gold bond, and subsequent coating of the construct with cationic polymer or physical adsorption of siRNA onto polymer coated AuNPs [Kim, H. J., et al., Precise engineering of siRNA delivery vehicles to tumors using polyion complexes and gold nanoparticles. ACS Nano, 2014. 8(9): p. 8979-91, Elbakry, A., et al., Layer-by-layer assembled gold nanoparticles for siRNA delivery. Nano Lett, 2009. 9(5): p. 2059-64]. While plethora of works have been reported on AuNPs as delivery system, its clinical translation has been a challenge. Some of the primary reasons for the failure of AuNPs for clinical translation include (i) limited drug loading, (ii) drug release issues and (iii) requirement of several surface layers for protection of biomolecules. Another important underlying drawback for such delivery systems include size of the particles. The particle size is one of the factors that dictates bioavailability and reticuloendothelial system clearance (RES) is a strong barrier for clinical translation [Alexis, F., et al., Factors affecting the clearance and biodistribution of polymeric nanoparticles. Mol Pharm, 2008. 5(4): p. 505-15.]. Also, the relevance of AuNP delivery system, specifically for the delivery of biomolecules such as siRNA, is totally diminished if siRNAs are directly exposed to external degradation factors present in human serum. To address the issue, layering on the surface of AuNPs for protecting siRNAs have been reported in literature Guo, S., et al., Enhanced gene delivery and siRNA silencing by gold nanoparticles coated with charge-reversal polyelectrolyte. ACS Nano, 2010. 4(9): p. 5505-11., Elbakry, A., et al., Layer-by-layer assembled gold nanoparticles for siRNA delivery. Nano Lett, 2009. 9(5): p. 2059-64., Song, W. J., et al., Gold nanoparticles capped with polyethyleneimine for enhanced siRNA delivery. Small, 2010. 6(2): p. 239-46]. However, the synthetic route for manufacturing in commercial scale, including purification, to obtain homogeneous compound deters its commercial viability and regulatory acceptance.

Advances in polymeric nanoparticles delivery systems (PNDS) has enabled delivery of small molecules to targeted site. Encapsulation and controlling the release of drugs provides synthetic design advantages of polymeric nanoparticle over metallic nananoparticles [Patil, Y. and J. Panyam, Polymeric nanoparticles for siRNA delivery and gene silencing, Int J Pharm, 2009. 367(1-2): p. 195-203.; Gary, D. J., N. Puri, and Y. Y. Won, Polymer-based siRNA delivery: perspectives on the fundamental and phenomenological distinctions from polymer-based DNA delivery, J Control Release, 2007. 121(1-2): p. 64-73]. For delivery of siRNA, the biomolecule is physically entrapped within polymeric nanoparticles and a cationic polymer, in some cases, is incorporated within the matrix for cytoplasmic delivery. Several works have been reported on the use of cationic polymers such as Polyethyleneimine (PEI) and Cyclodextrin for designing such systems [Kulkarni, A., et al., Pendant polymer:amino-beta-cyclodextrin:siRNA guest:host nanoparticles as efficient vectors for gene silencing, J Am Chem Soc, 2012. 134(18): p. 7596-9; Grayson, A. C., A. M. Doody, and D. Putnam, Biophysical and structural characterization of polyethylenimine-mediated siRNA delivery in vitro, Pharm Res, 2006. 23(8): p. 1868-76; Li, X., et al., A mesoporous silica nanoparticle—PEI—fusogenic peptide system for siRNA delivery in cancer therapy, Biomaterials, 2013, 34(4): p. 1391-401; Kanasty, R., et al., Delivery materials for siRNA therapeutics, Nat Mater, 2013. 12(11): p. 967-77.]. The use of PEI as a delivery system for in-vivo application is however highly restricted due to dose dependent toxicity. Also, with few exceptions like polymeric implant based therapy such as LODER, that has demonstrated significant tumor reduction and is currently in clinical trial phase for patients suffering from KRas mutation in pancreatic cancer, encapsulation of large molecules such as siRNA within PNDS for systemic delivery is synthetically challenging at large scale for clinical translation [Zorde Khvalevsky, E., et al., Mutant KRAS is a druggable target for pancreatic cancer. Proc Natl Acad Sci USA, 2013. 110(51): p. 20723-8.].

From various literatures reported, it is evident that oncogene knockdown using nanoparticle mediated delivery of siRNA lacks the potential for clinical translation. Apart from biological challenges including bio-distribution (modulating RES clearance and EPR effect), and clinical study design, the major impeding factor is the scalability. To circumvent the issue, other alternate methods have been explored for the delivery of siRNA including direct conjugation of siRNA to the delivery material, such as ligands. This technique enables control over composition and yields homogeneous formulations. The first of such systems used for targeting hepatocytes, called Dynamic PolyConjugates (DPC), was developed by Rozema et. al. by conjugating siRNA to amphipathic poly (vinyl ether) (PBAVE) through disulfide linkage and complexed with small molecule N-acetylgalactosamine (GalNAc) [Rozema, D. B., et al., Dynamic PolyConjugates for targeted in vivo delivery of siRNA to hepatocytes. Proc Natl Acad Sci USA, 2007. 104(32): p. 12982-7.]. In this case, for effective and stable delivery of siRNA, Polyethyelene Glycol (PEG) shielding was required. The system is highly specific towards liver, aided through GalNAc for targeting as well as for silencing. ZordeKhvalevsky, E., et al have reported another platform—LODER, as described previously [Zorde Khvalevsky, E., et al., Mutant KRAS is a druggable target for pancreatic cancer. Proc Natl Acad Sci USA, 2013. 110(51): p. 20723-8.]. LODER is intra-tumor implantable device that protects siRNA from serum degradation and releases siRNA over an extended period. However, the technology is limited to localized solid tumor with limited applicability to late stage cancer or metastases. Also, systemic delivery is not applicable for LODER technology.

In prior arts, siRNA is also directly linked to antibody without a macromolecule linker [Cuellar, T. L., et al., Systematic evaluation of antibody-mediated siRNA delivery using an industrial platform of THIOMAB-siRNA conjugates. Nucleic Acids Res, 2015. 43(2): p. 1189-203.]. However, the system as reported by Cuellar et al., comprises of antibody-siRNA conjugate linked through small molecule linker. Cuellar et al. devised antibody-siRNA conjugates (ARC) using THIOMAB technology that was earlier used for antibody drug conjugates. In this work, subsequent to modification of antibody by introducing a cysteine residue in the heavy chain, siRNA was covalently linked to the modified antibody through a small molecule linker Sulfo-SMCC. However, effective oncogene knockdown of this ARC platform was applicable only for specific systems. Systems with high antigen expression had highest gene silencing effects. In all the other cell lines explored in the work, weak or no knockdown was observed. The main issue of this platform was limited endosomal escape of ARCs post internalization for siRNAs to engage with RISC within the cytoplasm. Another reported work involves utilization of macromolecule linker for delivery of siRNA through antibody. The system reported in the work utilized fusion proteins containing positively charged peptide and single chain variable fragment (scFv) of antibody or Fab fragment [Peer, D., et al., Selective gene silencing in activated leukocytes by targeting siRNAs to the integrin lymphocyte function-associated antigen-1. Proc Natl Acad Sci USA, 2007. 104(10): p. 4095-100.]. Fc region of antibody is avoided in all such cases to avoid interaction of the fusion protein with complement and other molecules. Synthesis of the Fab or scFv antibody fragment with cationic peptide fusion protein is not only highly complex but lack of the Fc domain induces high risk of agglomeration during production and purification, thus limiting their utility for commercial application.

To move beyond nanoparticle mediated delivery and nonspecific cellular localization of siRNA, use of peptides and antibodies to deliver siRNA have been explored. Ligands such as peptides and aptamers have been commonly used for delivering siRNA [Meade, B. R. and S. F. Dowdy, Exogenous siRNA delivery using peptide transduction domains/cell penetrating peptides. Adv Drug Deliv Rev, 2007. 59(2-3): p. 134-40.; McNamara, J. O., 2nd, et al., Cell type-specific delivery of siRNAs with aptamer-siRNA chimeras. Nat Biotechnol, 2006. 24(8): p. 1005-15.]. While such systems are suitable for exploratory proof of concept studies, induction of antibody response, lack of commercial feasibility due to high costs and synthetic complexity mitigate its translation potential.

Thus there is an unmet need in the art to provide a delivery system with a single targeting ligand capable of conjugating with one or more macromolecules that act as a carrier for targeted delivery of an active agent such as biological molecule that can mitigate one or more of the shortcomings of the delivery systems of the existing art.

### Objective of the Invention

One object of the present disclosure is to provide delivery carriers for stable and targeted delivery of a biomolecule for example an siRNA, an shRNA, an oligonucleotide like DNA or RNA, or a peptide, which is required to enter cytoplasm or nucleus of the cells for therapeutic and diagnostic application(s).

It is another object of the present disclosure to provide biomarker targeting moiety for example an antibody, protein or peptide tagged with cationic macromolecule for example protein, or polymer to provide a macromolecule-biomarker targeting moiety complex.

It is yet another object of the present disclosure to provide a macromolecule-antibody complex for loading biomolecule such as but not limited to siRNA, either through physical entrapment mediated via electrostatic interaction, or through chemical linkage.

### SUMMARY OF THE INVENTION

The present disclosure in a general aspect provides a conjugate of a general Formula (1):

Aₙ-Lₓ-B_{y} Formula 1

wherein,
A is a biomarker targeting moiety selected from antibody, antibody fragment or peptide;
L is a macromolecule linker selected from one or more of protein, peptide, polypeptide or a polymer comprising at least one carboxyl group;
B is a biological molecule selected from one or more of nucleic acid(s) selected from siRNA, shRNA, or DNA; or a peptide;
n is 1;
x is 1-10;
y is 0-100; and
wherein A is linked to B through amide bond, disulfide bond or thio-ether bond; and B is attached to L through a thio-ether bond, a disulfide bond, or attached by electrostatic force of interaction, or physical entrapment or combination thereof.

In one aspect the present disclosure provides a conjugate of Formula (1):

An-Lx-By Formula 1

wherein,
A is a biomarker targeting moiety selected from antibody, antibody fragment or peptide;
L is a macromolecule linker selected from one or more of protein, peptide, polypeptide or a polymer comprising at least one carboxyl group;
n is 1;
x is 1-10;
y is 0; and
wherein A is linked to B through amide bond, disulfide bond or thio-ether bond.

In another aspect the present disclosure provides a conjugate of Formula (1):

An-Lx-By Formula 1

wherein,
A is a biomarker targeting moiety selected from antibody, antibody fragment or peptide;
L is a macromolecule linker selected from one or more of protein, peptide, polypeptide or a polymer comprising at least one carboxyl group;
B is a biological molecule selected from one or more of nucleic acid(s) selected from siRNA, shRNA, or DNA; or a peptide;
n is 1;
x is 1-10;
y is ≥1; and
wherein A is linked to B through amide bond, disulfide bond or thio-ether bond; and B is attached to L through a thio-ether bond, a disulfide bond, or attached by electrostatic force of interaction, or physical entrapment or combination thereof.

In an aspect the present disclosure provides a conjugate, wherein the antibody or antibody fragment comprises one or more of a free carboxyl group, a free amino group, or a free cystine group.

In an aspect the present disclosure provides a conjugate, wherein the peptide used as the biomarker targeting moiety comprises one or more of a free carboxyl group, a free amino group, or a free cystine group.

In an aspect the present disclosure provides a conjugate, wherein the macromolecule linker is a cationic molecule, or modified to a cationic molecule.

In an aspect the present disclosure provides a conjugate, wherein the macromolecule linker comprises at least two carboxyl groups, wherein one of the carboxyl group is modified to at least one amine group.

In an aspect the present disclosure provides a conjugate capable of delivering the biological molecule to the targeted site.

In an aspect the present disclosure provides a conjugate capable of delivering the biological molecule to the cytoplasm or the nucleus of the cells.

In an aspect the present disclosure provides a conjugate, wherein the conjugate is further labeled with fluorescent dye or radioactive label.

In an aspect the present disclosure provides a process for preparing a conjugate comprising entities as per Formula (1):

Aₙ-Lₓ-B_{y} Formula 1

wherein,
A is a biomarker targeting moiety selected from antibody, antibody fragment or peptide;
L is a macromolecule linker selected from one or more of protein, peptide, polypeptide, or a polymer comprising at least one carboxyl group;
B is a biological molecule selected from one or more of nucleic acid(s) selected from siRNA, shRNA, or DNA; or a peptide;
n is 1;
x is 1-10;
y is 0-100;
wherein the process comprises
   linking A to B chemically through amide bond, disulfide bond or thio-ether bond; and
   when y is ≥1, attaching B to L chemically through a thio-ether bond, a disulfide bond; or by electrostatic force of interaction, or physical entrapment, or combination thereof.

In one aspect the present disclosure provides a process for preparing a conjugate of Formula (1): An-Lx-By, wherein the n is 1, x is 1-10 and y is 0.

In another aspect the present disclosure provides a process for preparing a conjugate of Formula (1): An-Lx-By, wherein the n is 1, x is 1-10 and y is >1.

In another aspect the present disclosure provides a process for preparing a conjugate, wherein the linking of A to L is carried out by reacting amine present in A with amine reactive ester of L; or reacting amine reactive ester of A with amine of L; or reacting the thiol of cystine present in A with thiol of L.

In an aspect the present disclosure provides a process for preparing a conjugate, wherein the antibody or antibody fragment comprises one or more of a free carboxyl group, a free amino group, or a free cystine group.

In an aspect the present disclosure provides a process for preparing a conjugate, wherein the peptide used as the biomarker targeting moiety comprises one or more of a free carboxyl group, a free amino group, or a free cystine group.

In an aspect the present disclosure provides a process for preparing a conjugate, wherein the macromolecule linker is cationic molecule, or modified to cationic molecule.

In an aspect the present disclosure provides a process for preparing a conjugate, wherein the macromolecule linker comprises at least two carboxyl groups, wherein one of the carboxyl group is modified to at least one amine group.

In an aspect the present disclosure provides a process for preparing a conjugate, wherein the macromolecule linker is modified to introduce at least one functional group selected from thiol (—SH), or amine (—NH2).

In an aspect the amine group is introduced in the macromolecule through compound selected from the group consisting of ethylenediamine, spermidine, spermine, PEI, poly-lysine, arginine or combination thereof; or thiol reactivity is introduced through thiol-containing moieties selected from cystine, and/or thiol reactive moieties selected from gold residue or silver residue.

In an aspect the present disclosure provides a process for preparing a conjugate, wherein the B is attached to L to release and deliver a payload of the biological molecule mediated by mechanism selected from enzymatic cleavage, pH change, salt concentrations or temperature.

In an aspect the present disclosure provides a process for preparing a conjugate, wherein the B is attached to L by modification of the macromolecule linker L to comprise of at least one thiol-reactive moiety.

In an aspect the reactive ester is introduced through imide functional group of N-hydroxy imide ester, wherein the imide functional group is selected from succinimide or phthalimide; or reactive esters is introduced through hydroxy-benzotriazole esters or its derivatives.

In an aspect the macromolecule linker is selected from the group consisting of gelatin, collagen, chitosan, dextran, dextrin, polyethyleneimine (PEI), Ply(L-Lysine), polyglutamic acid, cationic polypeptide, analogue thereof, or derivative thereof.

In an aspect the macromolecule linker is gelatin or cationic gelatin.

In an aspect the gelatin molecule or cationic gelatin molecule is modified by synthetic modification selected from cleavage, chemical modification of carboxyl groups to amine groups, physical modification or combination thereof.

In an aspect the cationic gelatin is activated using N-(3-dimethylaminopropyl)-N′-ethylcarbodiimide and N-hydroxysuccinimide.

In an aspect the cationic gelatin is carried out at a pH below 5, preferably at a pH below 3 and more preferably at pH below 2.5.

In an aspect the concentration of the cationic gelatin is maintained below 100 mg/ml, preferably at a concentration below 50 mg/ml and more preferably at a concentration of 30 mg/ml.

In an aspect the concentration of biomarker targeting moiety is more than 1 mg/ml, preferably above 2 mg/ml and more preferably above 5 mg/ml.

In an aspect the pH of the biomarker targeting moiety is less than 11 prior to the addition of the macromolecule linker, preferably the pH is below 10, and more preferably the pH is below 9 but above 6.

In an aspect the reactions are performed using macromolecule linker and biomarker targeting moiety in a molar ratio greater than 1:5 and lesser than 1:100, preferably at a molar ratio between 1:20 and 1:80; and more preferably between 1:20 and 1:50.

In an aspect the process further comprises step(s) for purification of conjugate by removal of excess of macromolecule linker and/or biomarker targeting moiety

In an aspect the biological molecule is siRNA.

In another aspect the present disclosure provides use of conjugate of the present invention to release and deliver a payload of the biological molecule at a target site, and for gene knockdown.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form part of the present specification and are included to further illustrate aspects of the present disclosure. The disclosure may be better understood by reference to the drawings in combination with the detailed description of the specific embodiments presented herein.

FIG. 1: It shows a schematic representation of one of the exemplary embodiments of cAMB. the conjugate comprising of (i) an antibody, for example cetuximab or IgG in a preferred embodiment, (ii) one or more macromolecule cationic linker, for example gelatin in a preferred embodiment, linked to the antibody and (iii) one or more biomolecule, for example siRNA in a preferred embodiment, linked to each cationic linker.

FIG. 2: It details the route of synthesis wherein macromolecular linker such as gelatin is converted to reactive ester and is used as the starting material.

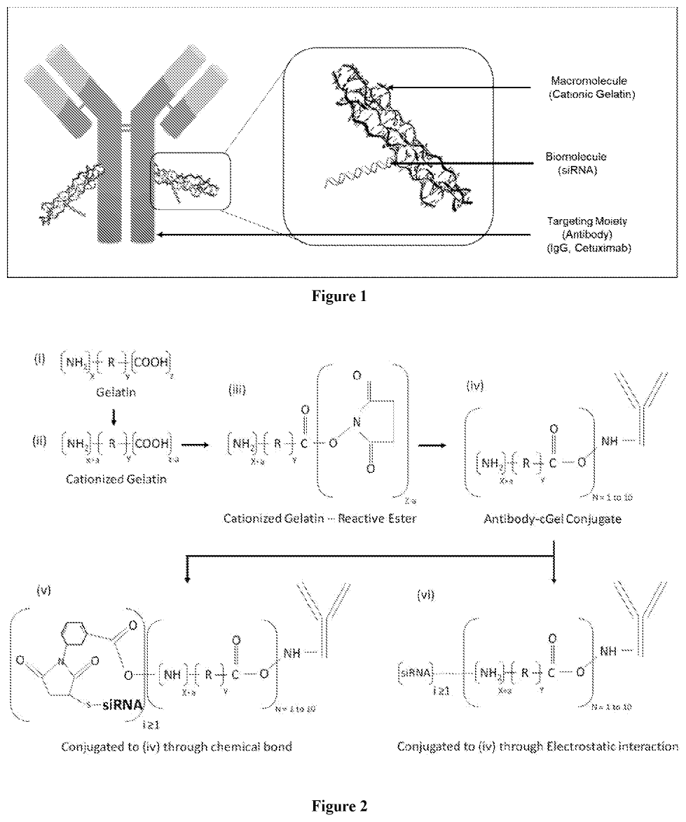

Step 1—carboxyl groups of gelatin are converted to amine groups partially in a process of cationization to obtain compound (ii).
Step 2—Residual carboxyl groups present in compound (ii) is modified to reactive ester group such as succinimide ester to form compound (iii).
Step 3—Amine reactive NHS functional group present in the compound (iii) reacts with lysine residues present in antibody to form compound (iv).
Step 4—siRNA is attached to compound (iv) either through chemical bond such as thiol-maleimide linkage to obtain compound (v) or through electrostatic interaction to obtain compound (vi).

FIG. 3: It details the route of synthesis wherein targeting moiety such as antibody is modified to reactive ester and used for conjugating with macromolecule linker such as gelatin.

Step 1—carboxyl groups of antibody are converted to reactive ester groups,
Step 2—carboxyl groups of gelatin are converted to amine groups partially in a process of cationization,
Step 3—compounds from step 1 and step 2 are reacted to obtain compound [vii],
Step 4—siRNA is attached to compound (vii) either through chemical bond such as thiol-maleimide linkage to obtain compound (viii) or through electrostatic interaction to obtain compound (ix).

FIG. 4: FIG. 4(a) shows the zeta potential of various molecules (i) IgG, (ii) IgG-cGelA225 complex (compound [iv.a]), IgG-cGelA225-siRNA complex (compound [v.a]), (iv) cetuximab, (v) cetuximab-cGelA225 complex (compound [iv.c]) and (vi) cetuximab-cGelA225-siRNA complex (compound [v.c]). Transition from neutral zeta potential of antibody to positively charged entity upon conjugating with cationized gelatin serves as a qualitative confirmatory tool for conjugation. (b) shows the change of zeta-potential of IgG-cGelA225 conjugate (compound [iv.a]) with varying pH. pH above 10 is generally preferred for attaching siRNA to the conjugate through electrostatic interaction due to its overall positive charge.

FIG. 5: it shows agarose gel electrophoresis, Reduced SDS-PAGE (14% gel), of compound [iv.c] under various siRNA conjugation and reaction conditions. Multiple distinct bands of the reduced conjugate confirms the presence of macromolecule attached to antibody. Bands with representation from 0 to 6 shows the number of cationized gelatin attached to light chain or heavy chain or both.

### Lane 1—Protein Marker

Lane 2—Reduced Ctb; Band near 50 KDa represents heavy chain and 25 KDa represents light chain of the antibody
Lane 3—Reduced compound [iv.c] synthesized at pH 6
Lane 4—Reduced compound [iv.c] synthesized at pH 7
Lane 5—Reduced compound [iv.c] synthesized at pH 8
Lane 6—Reduced compound [iv.c] synthesized at pH 9

FIG. 6: FIGS. 6(a) and (b) shows band shift through non-reducing SDS gel electrophoresis (14% gel) analysis for various compounds indicating the complex formation; (c) chromatography profile of purification of antibody-macromolecule conjugate.

FIG. 6(a):

### Lane 1—Purified IgG

Lane 2—Compound [iv.b]
Lane 3—Compound [iv.d]
Lane 4—Compound [vii.b]
Lane 5—Compound [vii.d]
Lane 6—Compound [iv.a]
Lane 7—Compound [iv.c]

FIG. 6(b):

### Lane 1—Blank

### Lane 2—Cetuximab

### Lane 3—Purified Cetuximab

### Lane 4—Blank

Lane 5—Compound [iv.d]

FIG. 7: Characterization and estimation of Antibody-Gelatin conjugates using analytical RP-HPLC. (7a) RP-HPLC profile of purified IgG sample shows the main peak produced at 5.8 Mins. (7b) RP-HPLC profile for compound [iv.b] shows a shift in the main peak from 5.8 Mins (unreacted IgG) to 8 Mins (conjugate).

FIG. 8: It shows gel retardation assay of compound [v.c] under various mole ratio of siRNA and compound [iv.c] as well as various reaction conditions. Thiol-siRNA (reduced) reacts with the conjugate with maleimide functionality efficiently at concentration above 5 mg/ml and preferably above 10 mg/ml.

Lane 1-15 mg/ml compound reacted with SS-siRNA at mole ratio of 4:1 (siRNA to compound [v.c])
Lane 2-15 mg/ml compound reacted with SS-siRNA at mole ratio of 2:1 (siRNA to compound [v.c])
Lane 3-15 mg/ml compound reacted with SS-siRNA at mole ratio of 1:1 (siRNA to compound [v.c])
Lane 4-10 mg/ml compound reacted with SS-siRNA at mole ratio of 4:1 (siRNA to compound [v.c])
Lane 5-10 mg/ml compound reacted with SS-siRNA at mole ratio of 2:1 (siRNA to compound [v.c])
Lane 6-10 mg/ml compound reacted with SS-siRNA at mole ratio of 1:1 (siRNA to compound [v.c])
Lane 7-5 mg/ml compound reacted with SS-siRNA at mole ratio of 4:1 (siRNA to compound [v.c])
Lane 8-5 mg/ml compound reacted with SS-siRNA at mole ratio of 2:1 (siRNA to compound [v.c])
Lane 9-5 mg/ml compound reacted with SS-siRNA at mole ratio of 1:1 (siRNA to compound [v.c])
Lane 10—siRNA control

FIG. 9: Colocalization of siRNA and Antibody-Gelatin complex. Native PAGE (ethidium bromide stained and Coomassie stained the same gel) for siRNA Antibody-macromolecule complex reaction samples with control. Conjugates containing siRNA can be visualized in both EtBr stain and Coomassie stain and is observed to be co-localized indicating the presence of siRNA conjugated to antibody-macromolecule complex.

### Lane 1—Protein Marker

Lane 2—compound [iv.a]
Lane 3—compound [v.a]
Lane 4—compound [v.c]
Lane 5—protein marker
Lane 6—compound [iv.c]
Lane 7—compound [vi.a]
Lane 8—compound [vi.c]

FIG. 10: Gel retardation assay of siRNA and compound [v.c] in 10% human serum and H23 cell lysate. The assay shows that the siRNA is stable in human serum and gets released from the compound in the presence of cell lysate. The release is possibly triggered by the degradation of gelatin by gelatinases present in cell lysate. It is known that cytoplasm contains various types of gelatinases.

Lane 1—compound [v.c]
Lane 2—compound [v.c] in 10% human serum for after 2 hours
Lane 3—compound [v.c] in 10% human serum at 37° C. after 0 hours
Lane 4—thiol-siRNA in 10% human serum after 0 hour
Lane 5—compound [v.c] in 10% human serum at 37° C. after 2 hours
Lane 6—thiol-siRNA in 10% human serum after 8 hour
Lane 7—compound [v.c] in H23 cell lysate at 37° C. after 2 hours

FIG. 11: It shows western blot analysis of H23 NSCLC cells treated with compound [v.c], lipofectamine transfected siRNA and for untreated H23 cells. Down-regulation of phosphorylated MEK and phosphorylated AKT proteins in the treated samples confirms KRas mRNA knockdown.

FIG. 12: It shows fluorescent microscopy image of H23 cells (fixed) treated with IgG and labelled with FITC conjugated secondary antibody under DAPI filter, FITC filter and their overlay. No prominent fluorescence was observed even at high exposure times (>4 Sec) indicating IgG does not bind to the cell surface receptors.

FIG. 13: It shows fluorescent microscopy image of H23 cells (fixed) treated with Ctb and labelled with FITC conjugated secondary antibody under DAPI filter, FITC filter and their overlay. Fluorescence was observed indicating Ctb binding to the cell surface receptors specifically.

FIG. 14: It shows fluorescent microscopy image of H23 cells (fixed) treated with compound [iv.d] and labelled with FITC conjugated secondary antibody under DAPI filter, FITC filter and their overlay. Fluorescence was observed indicating compound [iv.d] binding to the cell surface receptors specifically.

FIG. 15: It shows fluorescent microscopy image of H23 cells (fixed) treated with compound [iv.c] and labelled with FITC conjugated secondary antibody under DAPI filter, FITC filter and their overlay. Fluorescence was observed indicating compound [iv.c] binding to the cell surface receptors specifically.

FIG. 16: It shows fluorescent microscopy image of H23 cells (fixed) treated with compound [v.c] and labelled with FITC conjugated secondary antibody under DAPI filter, FITC filter and their overlay. Fluorescence was observed indicating incorporation of siRNA does not inhibit receptor biding and compound [v.c] binds to the cell surface receptors specifically.

FIG. 17: It shows fluorescent microscopy image of H23 cells (fixed) treated with compound [iv.a] and labelled with FITC conjugated secondary antibody under DAPI filter, FITC filter and their overlay. Fluorescence was observed, although not specific to the cell surface receptor indicating non-specific binding of the conjugate with cell surface receptors.

FIG. 18: It shows MTT assay to determine the cytotoxicity of compound [iv.c], compound [iv.a] and cetuximab on H23 NSCLC cells in the absence of siRNA and TKI (gefitinib). The results indicate negligible toxicity of the molecules and loss of viability of H23 cells was not observed.

FIG. 19: It shows MTT assay to determine the cytotoxicity of compound [v.c] and compound [v.a] on H23 NSCLC cells in the presence of siRNA and absence of TKI (gefitinib, 5 μM). The results indicate negligible toxicity of the molecules and loss of viability of H23 cells was not observed.

FIG. 20: It shows MTT assay to determine the cytotoxicity of compound [iv.c] and compound [iv.a] on H23 NSCLC cells in the absence of siRNA and presence of TKI (gefitinib, 5 μM). The results indicate negligible toxicity of the molecules and loss of viability of H23 cells was not observed.

FIG. 21: FIG. 21(a) shows MTT assay to determine the cytotoxicity of 0.5 μM cetuximab, 0.5 μM compound [v.c] and 0.5 μM compound [v.a] on H23 NSCLC cells in the presence of siRNA and presence of TKI (gefitinib, 0, 0.1, 0.5, 1 and 5 μM). The results indicate negligible toxicity of the cetuximab and compound [v.a]. However, approximately 90% loss of viability of H23 cells was observed in the case of H23 cells treated with compound [v.c] indicating the synergistic effect of gefitinib and mRNA knockdown leading to apoptosis of cells. FIG. 21(b) shows MTT assay to determine the cytotoxicity of gefitinib on H23 NSCLC cells. Gefitinib shows no toxicity on H23 cells up to a concentration of 25 μM. At 50 μM gefitinib concentration, 70% loss of viability of H23 cells was observed.

FIG. 22: Gel retardation assay of compound [vi.c]; siRNA was added to 5 mg/ml of conjugate at a 2:1 mole ratio (siRNA:Conjugate) at various pH. It is preferable that the pH of the solution is above 10 for efficient binding.

Lane 1—Physical entrapment of siRNA to compound [vi.c] at pH 5
Lane 2—Physical entrapment of siRNA to compound [vi.c] at pH 6
Lane 3—Physical entrapment of siRNA to compound [vi.c] at pH 7
Lane 4—Physical entrapment of siRNA to compound [vi.c] at pH 8
Lane 5—Physical entrapment of siRNA to compound [vi.c] at pH 10

FIG. 23: FIG. 23(a) Gel retardation assay of compound [vi.c] at a mole ratio of siRNA to compound of 1:1 at various concentrations.

Lane 1—Physical entrapment of siRNA to compound [vi.c] at a compound concentration of 0 mg/ml
Lane 2—Physical entrapment of siRNA to compound [vi.c] at a compound concentration of 0.25 mg/ml
Lane 3—Physical entrapment of siRNA to compound [vi.c] at a compound concentration of 0.5 mg/ml
Lane 4—Physical entrapment of siRNA to compound [vi.c] at a compound concentration of 1 mg/ml
Lane 5—Physical entrapment of siRNA to compound [vi.c] at a compound concentration of 5 mg/ml
Lane 6—Physical entrapment of siRNA to compound [vi.c] at a compound concentration of 10 mg/ml

FIG. 23 (b) is a Densitometry analysis of (a), detailing the levels of bound siRNA with varying concentration of compound.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively, and any and all combinations of any or more of such steps or features.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Various terms are used herein. To the extent a term used in a claim is not defined below, it should be given the broadest definition persons in the pertinent art have given that term as reflected in printed publications and issued patents at the time of filing.

### Definitions and Abbreviations

In the text, wherever used the following meanings are used for the below abbreviations:

siRNA refers to Short Interfering RNA

NP refers to nanoparticle

AuNP refers to gold nanoparticles

PNDS refers to polymeric nanoparticles delivery systems

PEI refers to Polyethyleneimine

LODER refers to Localized Delivery of siRNA

KRas refers to Kirsten Rat Sarcoma viral oncogene homolog

RES refers to Reticulo Endothelial System

EPR refers to Enhanced Permeability and Retention

DPC refers to Dynamic PolyConjugates

PBAVE refers to Amphipathic poly vinyl ether

GalNAc refers to N-acetylgalactosamine

PEG refers to Polyethyelene Glycol

ARC refers to Antibody-siRNA conjugates

RISC refers to RNA-induced silencing complex

Fc—Antibody Constant Fragment

Fv refers to Antibody Variable Fragment

Fab refers to Antibody antigen binding Fragment

scFv refers to single chain variable fragment

DNA refers to Deoxyribonucleic Acid

RNA refers to ribonucleic Acid

cAMB: Antibody-Macromolecule linker-Biomolecule complex

GLP-1 refers to Glucagon-like peptide 1

EGFR refers to epidermal growth factor receptor

IgG refers to Immunoglobulin G

CRISPR refers to Clustered Regularly Interspaced Short Palindromic Repeats

shRNA refers to Short ribonucleic Acid

NSCLC refers to Non small cell lung cancer cells

EDC refers to 1-Ethyl-3-(3-dimethylaminopropyl) carbodiimide

NHS refers to N-hydroxysuccinimide

Sulfo-MBS refers to m-maleimidobenzoyl-N-hydroxysulfosuccinimide ester

ss-siRNA refers to thiol-reactive siRNA

CTB refers to Cetuximab

GEL refers to Gelatin

RP-HPLC refers to Reverse Phase—High Performance Liquid Chromatography

DI Water refers to De-Ionised Water

EtBr refers to ethidium bromide

MTT refers to 3-(4,5-Dimethylthiazol-2-yl)-2,5-diphenyltetrazolium bromide

TKI refers to Tyrosine Kinase Inhibitor

HCl refers to Hydrochloric Acid

MES refers to 2-(N-Morpholino)ethanesulfonic acid

PBS refers to phosphate buffer solution

SDS PAGE refers to sodium dodecyl sulfate polyacrylamide gel electrophoresis

RT refers to Room Temperature

EDTA—Ethylenediaminetetraacetic acid

cGelA225 refers to Cationised Gelatin TypeA bloom225

cGelA110 refers to Cationised Gelatin TypeA bloom110

Ctb-cGelA225 refers to EDC/NHS activated Cationised Gelatin TypeA bloom225 conjugated with Cetuximab, also referred to as compound [iv.c]

Ctb-cGelAl10 refers to EDC/NHS activated Cationised Gelatin TypeA bloom110 conjugated with Cetuximab, also referred to as compound [iv.d]

IgG-cGelA225 refers to EDC/NHS activated Cationised Gelatin TypeA bloom225 conjugated with ImmunoglobulinG, also referred to as compound [iv.a]

IgG-cGelA110 refers to EDC/NHS activated Cationised Gelatin TypeA bloom110 conjugated with ImmunoglobulinG, also referred to as compound [iv.b]

IgG-cGelA225-siRNA(thiol) refers to EDC/NHS activated Cationised Gelatin TypeA bloom225 conjugated with ImmunoglobulinG, chemically attached to siRNA through thio-ether bond, also referred to as compound [v.a]

IgG-cGelA110-siRNA(thiol) refers to EDC/NHS activated Cationised Gelatin TypeA bloom110 conjugated with ImmunoglobulinG, chemically attached to siRNA through thio-ether bond, also referred to as compound [v.b]

Ctb-cGelA225-siRNA(thiol) refers to EDC/NHS activated Cationised Gelatin TypeA bloom225 conjugated with Cetuximab, chemically attached to siRNA through thio-ether bond, also referred to as compound [v.c]

Ctb-cGelA110-siRNA(thiol) refers to EDC/NHS activated Cationised Gelatin TypeA bloom110 conjugated with Cetuximab, chemically attached to siRNA through thio-ether bond, also referred to as compound [v.d]

IgG-cGelA225-siRNA refers to EDC/NHS activated Cationised Gelatin TypeA bloom225 conjugated with ImmunoglobulinG, attached to siRNA through Electro static interaction, also referred to as compound [vi.a]

IgG-cGelA110-siRNA refers to EDC/NHS activated Cationised Gelatin TypeA bloom110 conjugated with ImmunoglobulinG, attached to siRNA through Electro static interaction, also referred to as compound [vi.b]

Ctb-cGelA225-siRNA refers to EDC/NHS activated Cationised Gelatin TypeA bloom225 conjugated with Cetuximab, attached to siRNA through Electro static interaction, also referred to as compound [vi.c]

Ctb-cGelA110-siRNA refers to EDC/NHS activated Cationised Gelatin TypeA bloom110 conjugated with Cetuximab, attached to siRNA through Electro static interaction, also referred to as compound [vi.d]

IgG(A)-cGelA225 refers to EDC/NHS activated ImmunoglobulinG Conjugated with Cationised Gelatin TypeA bloom225, also referred to as compound [vii.a]

IgG(A)-cGelA110 refers to EDC/NHS activated ImmunoglobulinGConjugated with Cationised Gelatin TypeA bloom110, also referred to as compound [vii.b]

Ctb(A)-cGelA225 refers to EDC/NHS activated CetuximabConjugated with Cationised Gelatin TypeA bloom225, also referred to as compound [vii.c]

Ctb(A)-cGelA110 refers to EDC/NHS activated CetuximabConjugated with Cationised Gelatin TypeA bloom110, also referred to as compound [vii.d]

IgG(A)-cGelA225-siRNA(thiol) refers to EDC/NHS activated ImmunoglobulinG Conjugated with Cationised Gelatin TypeA bloom225, chemically attached to siRNA through thiorefers to ether bond, also referred to as compound [viii.a]

IgG(A)-cGelA110-siRNA(thiol) refers to EDC/NHS activated ImmunoglobulinG Conjugated with Cationised Gelatin TypeA bloom110, chemically attached to siRNA through thio-ether bond, also referred to as compound [viii.b]

Ctb(A)-cGelA225-siRNA(thiol) refers to EDC/NHS activated Cetuximab Conjugated with Cationised Gelatin TypeA bloom225, chemically attached to siRNA through thio-ether bond, also referred to as compound [viii.c]

Ctb(A)-cGelA110-siRNA(thiol) refers to EDC/NHS activated Cetuximab Conjugated with Cationised Gelatin TypeA bloom110, chemically attached to siRNA through thio-ether bond, also referred to as compound [viii.d]

IgG(A)-cGelA225-siRNA refers to EDC/NHS activated ImmunoglobulinG Conjugated with Cationised Gelatin TypeA bloom225, attached to siRNA through Electro static interaction, also referred to as compound [ix.a]

IgG(A)-cGelA110-siRNA refers to EDC/NHS activated ImmunoglobulinG Conjugated with Cationised Gelatin Type A bloom110, attached to siRNA through Electro static interaction, also referred to as compound [ix.b]

Ctb(A)-cGelA225-siRNA refers to EDC/NHS activated Cetuximab Conjugated with Cationised Gelatin TypeA bloom225, attached to siRNA through Electro static interaction, also referred to as compound [ix.c]

Ctb(A)-cGelA110-siRNA refers to EDC/NHS activated Cetuximab Conjugated with Cationised Gelatin TypeA bloom110, attached to siRNA through Electro static interaction

FITC refers to Fluorescein isothiocyanate

A₂₈₀ refers to Absorbance at 280 nm

KDa refers to Kilo Dalton

cAMB refers to Antibody-Macromolecule-Biomolecule conjugate

The term “biomolecule” as used herein refers to and is used interchangeably with “biological molecule” which refers to one or more of nucleic acid(s) selected from siRNA, shRNA, or DNA; or a peptide.

The term “Cationised” as used herein in context of macromolecule, or gelatin as an exemplary macromolecule, is used interchangeably with “Cationic” and refers to a molecule which is cationic in nature i.e. possessing positive charge, or a molecule is modified to possess positive charge.

The detailed description of the antibody-biomolecule conjugate also referred to as the Antibody-Macromolecule-Biomolecule conjugate (cAMB) interchangeably is given below.

In general the present invention relates to a conjugate for delivery of biomolecules using a cationic macromolecule as a linker between the biomolecule and biomarker targeting moiety, and the process of making the conjugate.

In an embodiment the present disclosure provides a conjugate of a general Formula (1):

Aₙ-Lₓ-B_{y} Formula 1

wherein,
A is a biomarker targeting moiety selected from antibody, antibody fragment or peptide;
L is a macromolecule linker selected from one or more of protein, peptide, polypeptide or a polymer comprising at least one carboxyl group;
B is a biological molecule selected from one or more of nucleic acid(s) selected from siRNA, shRNA, or DNA; or a peptide;
n is 1;
x is 1-10;
y is 0-100; and
wherein A is linked to B through amide bond, disulfide bond or thio-ether bond; and B is attached to L through a thio-ether bond, a disulfide bond, or attached by electrostatic force of interaction, or physical entrapment or combination thereof.

In an embodiment the present disclosure provides a conjugate of Formula (1):

An-Lx-By Formula 1

wherein,
A is a biomarker targeting moiety selected from antibody, antibody fragment or peptide;
L is a macromolecule linker selected from one or more of protein, peptide, polypeptide or a polymer comprising at least one carboxyl group;
n is 1;
x is 1-10;
y is 0; and
   wherein A is linked to B through amide bond, disulfide bond or thio-ether bond.

In an embodiment the present disclosure provides a conjugate of Formula (1):

An-Lx-By Formula 1

wherein,
A is a biomarker targeting moiety selected from antibody, antibody fragment or peptide;
L is a macromolecule linker selected from one or more of protein, peptide, polypeptide or a polymer comprising at least one carboxyl group;
B is a biological molecule selected from one or more of nucleic acid(s) selected from siRNA, shRNA, or DNA; or a peptide;
n is 1;
x is 1-10;
y is ≥1; and
   wherein A is linked to B through amide bond, disulfide bond or thio-ether bond; and B is attached to L through a thio-ether bond, a disulfide bond, or attached by electrostatic force of interaction, or physical entrapment or combination thereof.

The 3 entities of the conjugate are either (i) chemically linked to each other according to the general formula 1 or (ii) A and L is chemically linked while B is attached through electrostatic attraction or physical entrapment to the conjugate. In the case of (i), amines present in A is linked to amine reactive ester of L or vice versa, through amide bond, or through disulfide bond or through thio-ether bond, whereas, B is linked to L either through a thio-ether bond or disulfide bond. In the case of (ii), A and L are linked same as case (i) while B is attached by electrostatic force of interaction due to differential charge, macromolecule condensation and entrapment or by van-der-wall's force of interaction.

One of the embodiments of this invention relates to biomarker targeting moiety-biomolecule conjugates wherein a biomolecule is linked to a biomarker targeting moiety through a macromolecule. Such a delivery system protects biomolecules such as siRNA from degradation and is aimed to provide better bioavailability and effective gene knockdown.

In one of the embodiments of the invention, targeting of the conjugate to cells is achieved through biomarker-specific antibodies or peptides.

In yet another embodiment, introduction of a macromolecule linker, capable of carrying a biomolecule, into any biomarker targeting moiety such as but not limited to antibody or peptide is reported. The antibodies may exist as whole monoclonal antibody, whole polyclonal antibody, antigen binding domain (Fab) fragment single arm, antigen binding domain (F(ab′)2) fragment two arms, single chain variable fragment (scFv), dimeric single chain variable fragment (di-scFv), single domain antibody fragment (sdAb), bispecific antibodies and their fragments such as but not limited to trifunctional antibody. Examples of such antibodies include but not limited to adalimumab, bevacizumab, cetuximab, rituximab, infliximab, abciximab, trastuzumab, ranibizumab and fragments of such antibodies.

It yet another embodiment, such antibody or antibody fragment has at least one free carboxyl group or one free amino group or one free cystine group or combination thereof.

In yet another embodiment, such functional groups may be present in the constant region of the antibody fragment, variable region of the antibody fragment or in both regions.

In yet another embodiment, it is found that coupling of macromolecule to antibody, may fully or partially affect the active domains of such antibody responsible for binding to ligands such as cell surface receptors. However, it is preferred that such alteration does not bring about a substantial change in the binding affinity of the modified antibody towards ligands.

In yet another embodiment, peptides and proteins can be used as the biomarker targeting moiety. When the peptide is used as the biomarker targeting moiety, said peptide comprises one or more of a free carboxyl group, a free amino group, or a free cystine group. Peptides that may be used as a biomarker targeting moiety can be selected from the group consisting of but not limited to corticotropin-releasing factor, parathyroid hormone, ACTH, angiotensin, calcitonin, enterogastrin, somatostatin, somatotropin, exendin and analogues thereof, insulin and analogues thereof, insulin-like growth factor-1, glucagon and analogues thereof, prolactin, thyroid stimulating hormones, pituitary adenylate cyclase activating peptide, secretin, somatomedin, glucagon-like peptide-1 and analogues thereof, glucagon-like peptide-2 and analogues thereof, insulin-like growth factor-2, gastric inhibitory peptide, growth hormone-releasing factor, thrombopoietin, erythropoietin, hypothalamic releasing factors, endorphins, enkephalins, vasopressin, oxytocin, opioids and analogues thereof, superoxide dismutase, interferon, asparaginase, arginase, adenosine deaminase, ribonuclease and arginine deaminase, which have relatively low blood residence time can be used to target specific receptors for activation and subsequent delivery of biomolecules.

In one embodiment, the macromolecule linker is generally cationic in nature or modified to a cationic molecule.

In an embodiment the macromolecule linker comprises at least two carboxyl groups, wherein one of the carboxyl group is modified to at least one amine group.

In an embodiment the macromolecule linker is modified to introduce at least one functional group selected from thiol (—SH), or amine (—NH₂).

In another embodiment, it is preferable that the macromolecule linkers may or may not possess charge reversable properties at pH ranging from 3-8 (FIG. 4).

In a further embodiment, the macromolecule linker may be a cationic protein, cationic peptide, cationic polymer derived through synthetic means or natural process, modified proteins, modified polymers, modified peptides and cell penetrating peptides or combination thereof.

In yet another embodiment, it is also preferable that such macromolecule is negatively charged or positively charged but can be modified to cationic molecule or modified to obtain relatively higher positive charge respectively through synthetic modification, such as but not limited to cleavage, chemical modification, physical modification or combination thereof.

In yet another embodiment, it is preferable that the linker or the targeting moiety-macromolecule conjugate is also able to release the payload such as a biomolecule.

In yet another embodiment, it is preferable that the linker or the targeting moiety-macromolecule conjugate is also able to release the payload such as a biomolecule. to the targeted site. In yet another embodiment, it is preferable that the linker or the targeting moiety-macromolecule conjugate is also able to release the payload such as a biomolecule to the cytoplasm or the nucleus of the cells.

In yet another embodiment, the release of the payload such as siRNA can be mediated via mechanisms such as but not limited to enzymatic cleavage, pH change, salt concentrations and temperature.

In one embodiment of the invention, the macromolecule linker includes proteins, polymers and peptides which has at least two carboxyl groups out of which, one carboxyl group can be modified to at least one amine group in a process of cationization or has at least one carboxyl group and one amine group. Examples of such macromolecule linkers include but is not limited to gelatin, collagen, chitosan, dextran, dextrin, Polyethyleneimine (PEI), Ply(L-Lysine), Polyglutamic acid, their respective analogues and their respective derivatives thereof.

In an embodiment the macromolecule linker is a gelatin or a cationic gelatin.

In yet another embodiment, the macromolecule linker can be modified chemically to incorporate suitable functional groups such as thiol (—SH) and amine (—NH₂) groups

In yet another embodiment, the modification includes but is not limited to introduction of amine residues to the carboxyl groups of the macromolecule linker.

Examples of such amine containing compounds include but not limited to ethylenediamine, spermidine, spermine, PEI, Poly-lysine, Arginine or combination thereof.

In another embodiment, it is also desirable that modifications in the macromolecular linker is carried out to introduce other functionalities such as but not limited to maleimide reactivity, or thiol reactivity through introduction of thiol-containing moieties, such as cystine, and/or thiol reactive moieties such as gold residue or silver residue.

In a preferred embodiment of the present invention, gelatin and/or its derivatives is used as the macromolecule linker. As a cationic polymer, gelatin is known to mediate proton sponge effect and enable endosomal escape. Also, gelatin degrades within the cells due to the presence of gelatinases. Secondly, gelatin in conjunction with the antibody acts as a protective globule for siRNA and restricts siRNA degradation.

In a further preferred embodiment, gelatin type A is used as macromolecule linker. Even more preferably, cationized gelatin obtained through modification of carboxyl groups to amine groups using ethylenediamine is utilized. The increase in charge is measured using zeta potential which confirms cationization (FIG. 4).

Such cationized gelatin possesses high positive charge and is used for binding of siRNA through electrostatic means and through chemical conjugation. In addition, such positive nature of the gelatin induces possible endosomal escape releasing the siRNA to the cytoplasm of the cells.

In yet another embodiment, Immunoglobulin G (IgG) is used as a control targeting ligand for applicable demonstration.

In one embodiment of the present invention, it is preferable that the biomolecule comprises of nucleic acid payloads such as, but not limited to siRNA, DNA, Crispr-Cas9 system and shRNA.

In yet another embodiment, delivery of siRNA to mediate oncogene knockdown is effectively achieved through the approach mentioned for therapeutic action.

In yet another embodiment, the conjugate system is also capable of delivering small molecules such as chemotherapeutic agent for targeted delivery.

In an embodiment the conjugate is further labeled with fluorescent dye or radioactive label.

In yet another embodiment, such conjugate systems can be used for diagnostic purposes through fluorescent dye labelling or radiolabeling.

In another embodiment of the invention, a construct, herein referred as cAMB (Antibody Macrolinker Biomolecule complex), is used for delivery of siRNA_{KRas} to adenocarcinoma of non-small cell lung cancer (NSCLC) harboring KRas G12C mutation (NCI-H23 cells) and gene knockdown.

In another embodiment, NCI-A549 harboring G12V mutation is used as control cell line.

In yet another embodiment, Cetuximab—a monoclonal antibody, is used as the targeting moiety for targeting overexpressing human epidermal growth factor receptor (EGFR) present in NCI-H23 cells.

In one embodiment, cAMB comprising of cationic gelatin molecules are attached to Cetuximab through amide bonds.

In an embodiment the cAMB conjugate comprises: (i) an antibody, for example cetuximab or IgG in a preferred embodiment, (ii) one or more macromolecule cationic linker, for example gelatin in a preferred embodiment, linked to the antibody and (iii) one or more biomolecule, for example siRNA in a preferred embodiment, linked to the cationic linker.

In an embodiment the present disclosure provides a process for preparing a conjugate comprising entities as per Formula (1):

An-Lx-By Formula 1

wherein,

A is a biomarker targeting moiety selected from antibody, antibody fragment or peptide;
L is a macromolecule linker selected from one or more of protein, peptide, polypeptide, or a polymer comprising at least one carboxyl group;
B is a biological molecule selected from one or more of nucleic acid(s) selected from siRNA, shRNA, or DNA; or a peptide;
n is 1;
x is 1-10;
y is 0-100;
wherein the process comprises
   linking A to B chemically through amide bond, disulfide bond or thio-ether bond; and
   when y is ≥1, attaching B to L chemically through a thio-ether bond, a disulfide bond; or by electrostatic force of interaction, or physical entrapment, or combination thereof.

In one embodiment the present disclosure provides a process for preparing a conjugate of Formula (1): An-Lx-By, wherein the n is 1, x is 1-10 and y is 0.

In another aspect the present disclosure provides a process for preparing a conjugate of Formula (1): An-Lx-By, wherein the n is 1, x is 1-10 and y is ≥1.

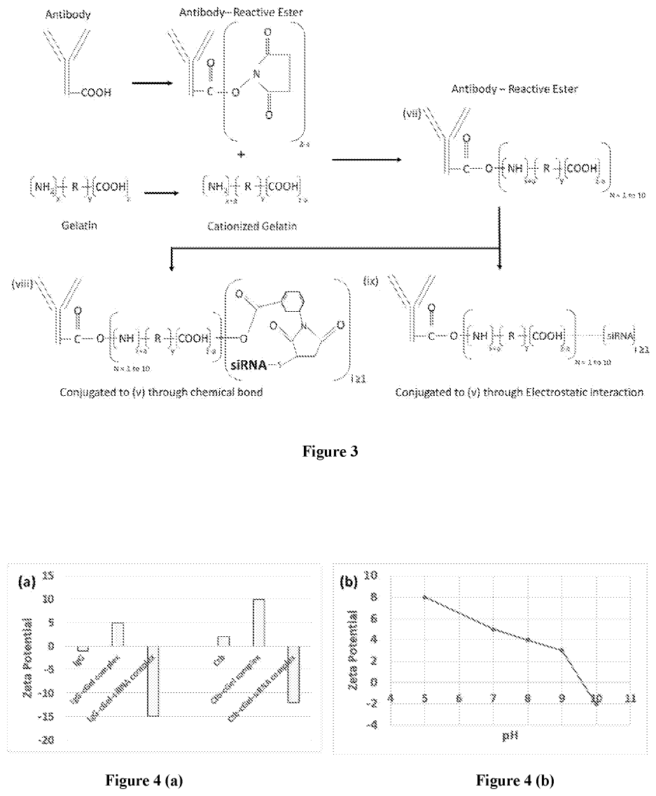

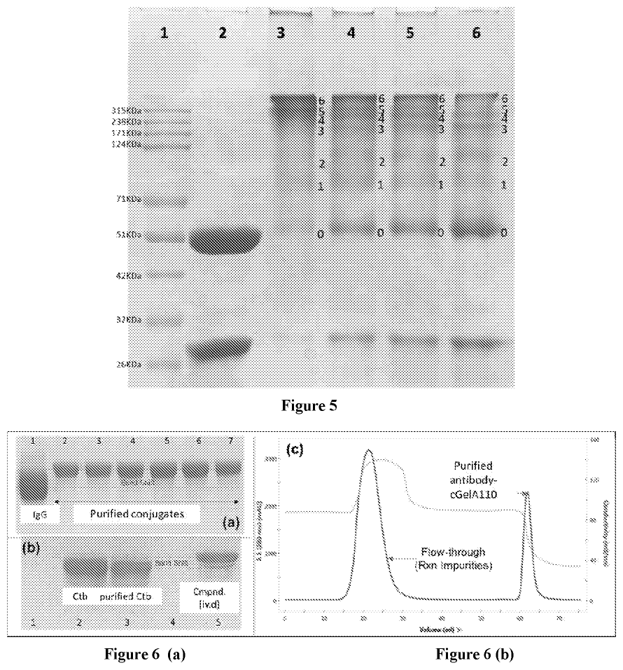

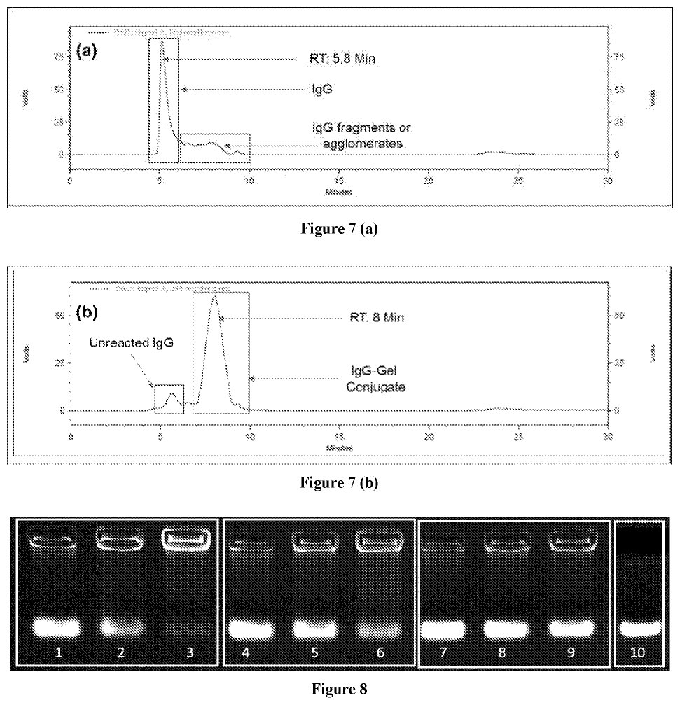

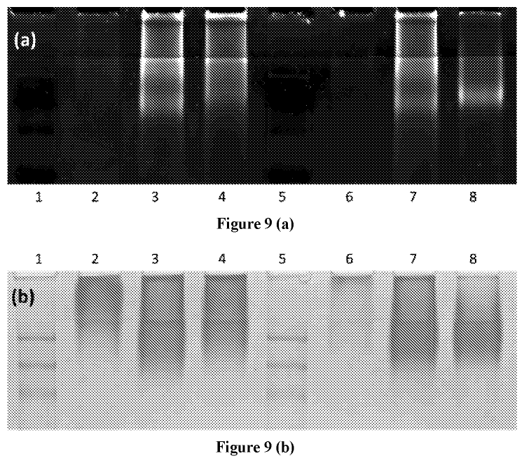

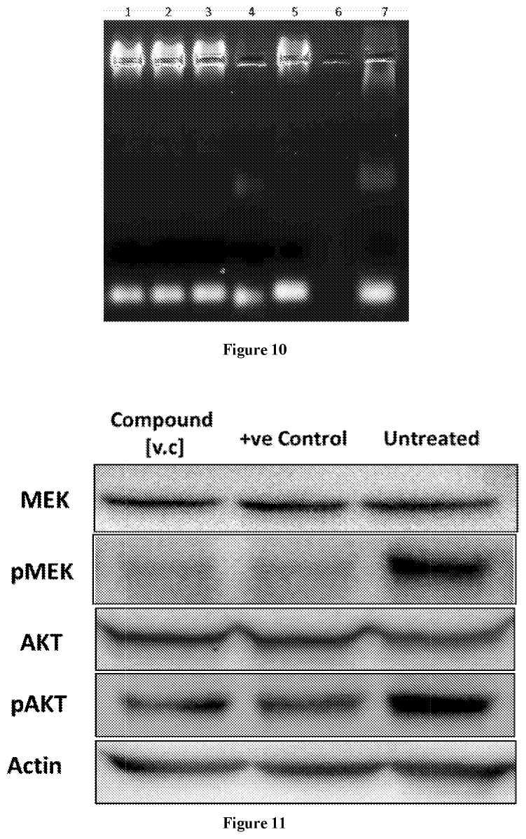

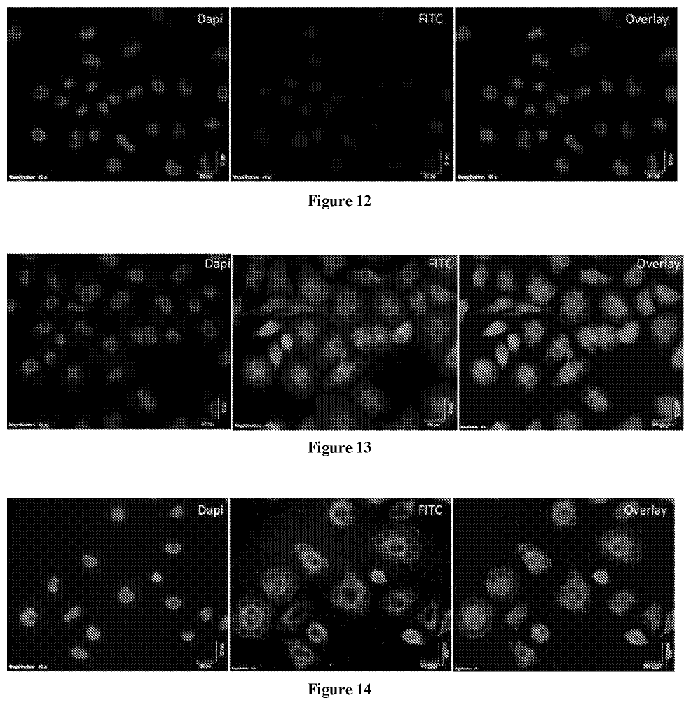

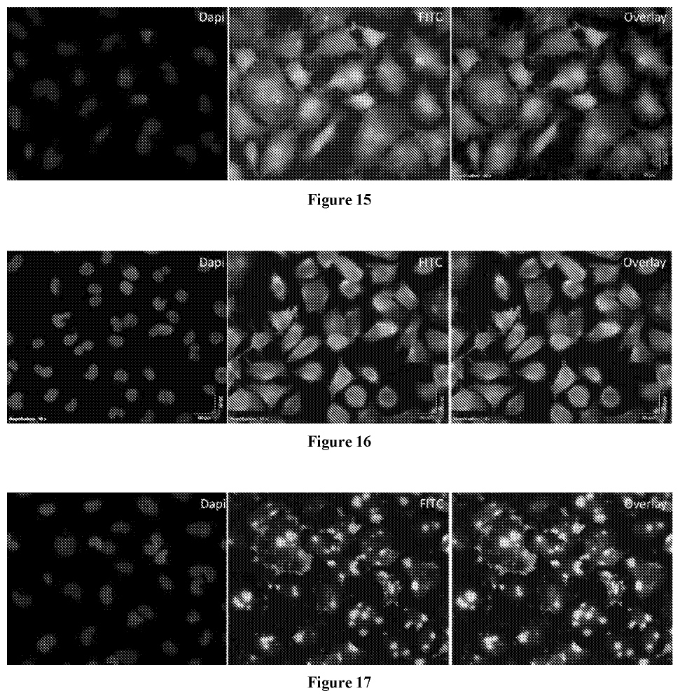

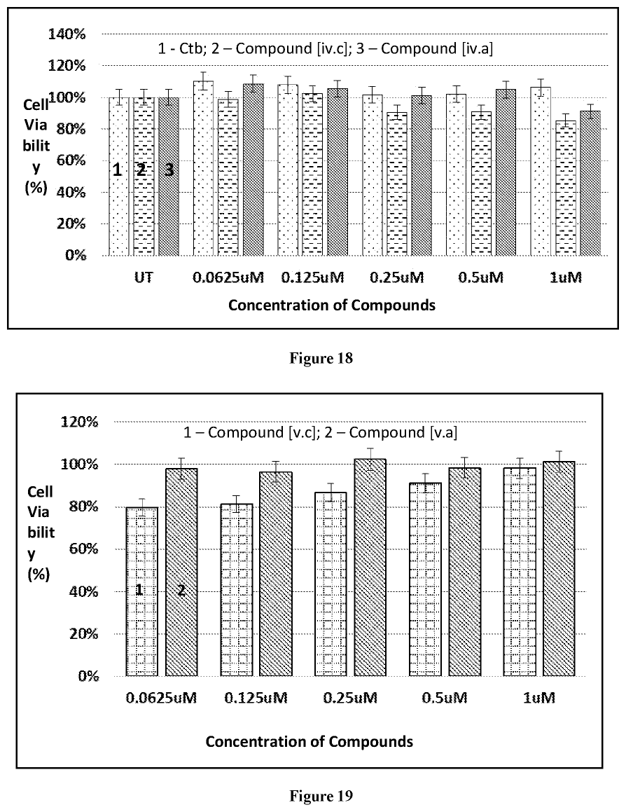

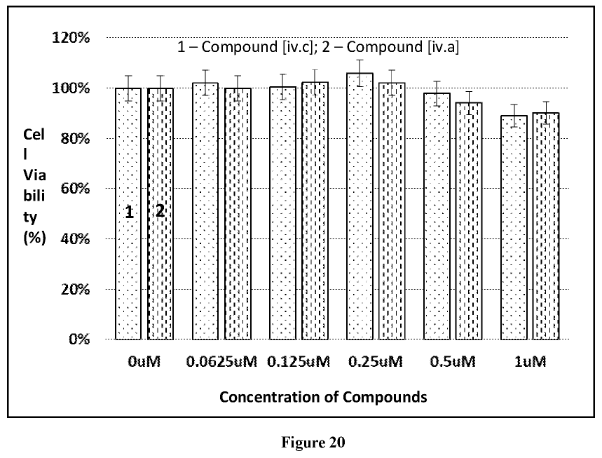

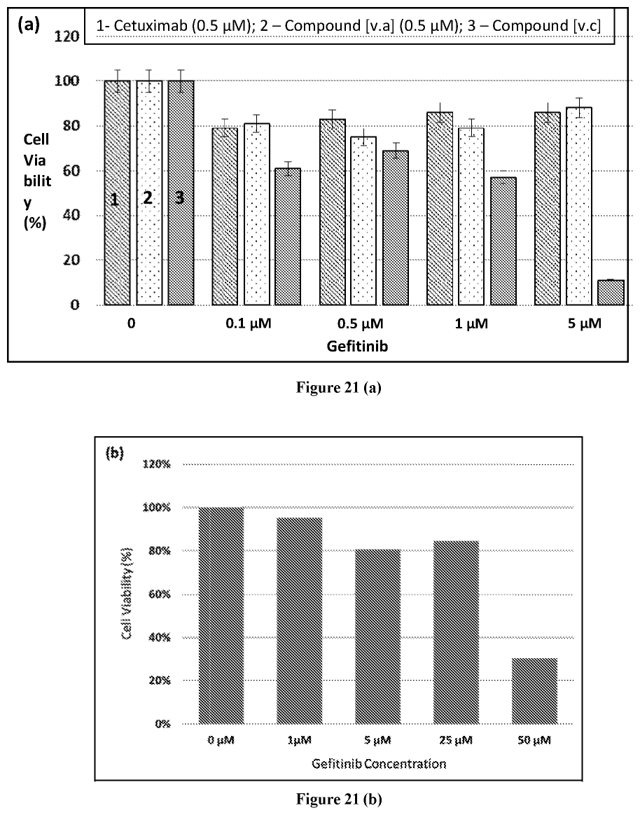

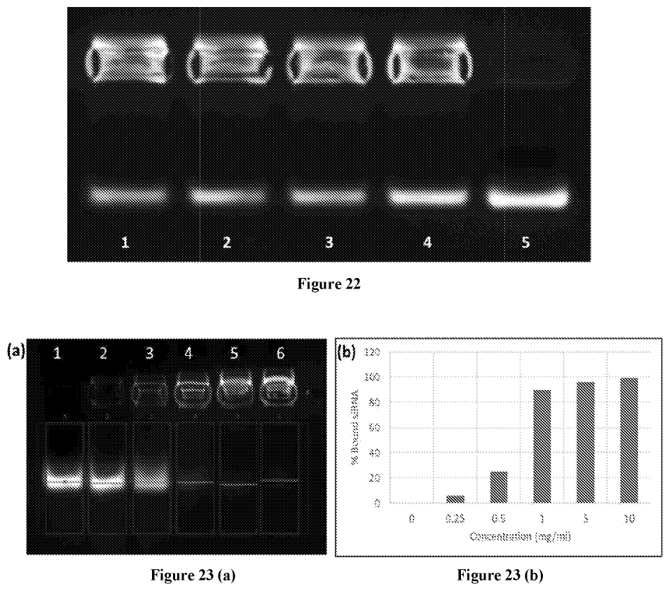

In one embodiment of the invention, the conjugation of the macromolecule to the antibody is carried out using two methodologies namely (i) modification of the macromolecule linker to amine reactive ester (FIG. 2) or (ii) modification of lysine residues of antibody to amine reactive ester or thiol-reactive moiety (FIG. 3).

In another embodiment the present disclosure provides a process for preparing a conjugate, wherein the linking of A to L is carried out by reacting amine present in A with amine reactive ester of L; or reacting amine reactive ester of A with amine of L; or reacting the thiol of cystine present in A with thiol of L.

In another embodiment, esterification of carboxylic acid group of macromolecule or antibody is carried out.

In yet another embodiment, it is preferable that the esters are imide functional group of reactive N-Hydroxy imide esters. The imide functional group can include but not limited to succinimide or phthalimide. Reactive esters can also be introduced through hydroxy-benzotriazole esters or its derivatives.

It is found that esterification of carboxyl group for reactivity towards amine moiety is complex when such macromolecule linkers contain amine functional groups in addition to carboxyl groups. In such cases, during the process of esterification, crosslinking occurs and reactivity towards amines of targeting ligands is lost.

In one of the embodiments, a carboxyl group of macromolecule containing amine groups, was esterified to form N-Hydroxysuccinimide ester under suitable conditions to ensure no cross linking occurs.

In a preferred embodiment of the invention, low as well as high molecular weight cationic gelatin are utilized as the linker. Cationic gelatin comprises of both carboxyl and amine functional groups required for protein chain modification and conjugation. Cationic nature of the protein imparts positive charge to the material, a property that mediates endosomal escape of the biomolecule.

Modification of carboxyl functional groups of gelatin to form reactive esters has been reported earlier. However, these reports are for the esterification of gelatin nanoparticles and not of gelatin. It is important to know that in the case of such nanoparticles, the amine residues are crosslinked using cross linking agents such as glutaraldehyde and therefore, the activation is simple as cross reactivity between amines and esterified carboxyl groups is avoided.

It is important to note that the linking of cationic macromolecule containing amine and carboxyl moiety to antibody is not reported. Specifically, conversion of carboxyl groups present in a macromolecule such as protein or polymer to reactive esters such as Succinimide ester is extremely challenging when amine moieties are also present in the same macromolecule as the molecule would tend to cross-link with each other. Specifically, in the case of gelatin, or more specifically in the case of cationized gelatin, the cross linking is highly pronounced and gelation occurs rendering the activated cationized gelatin non-functional for conjugating to antibodies or peptides.

In one aspect of the present invention, upon cationization and processing of cationized gelatin, activation of the cationized gelatin using N-(3-Dimethylaminopropyl)-N′-ethylcarbodiimide and N-Hydroxysuccinimide chemistry is carried out.

In one embodiment of the aspect, the concentration of the cationized gelatin is maintained below 100 mg/ml, preferably at a concentration below 50 mg/ml and more preferably at a concentration of 30 mg/ml.

In another embodiment, it is preferable to carry out the activation of the cationized gelatin at a pH below 5, contrary to conventional methods where activation is generally carried out at a pH ranging from pH 5 to pH 7.

In yet another embodiment, it is preferable to activate the cationized gelatin at a pH below 5, preferably at a pH below 3 and more preferably at pH below 2.5.

In yet another embodiment, the cross-linking of cationized gelatin during esterification was avoided. The cross linking was visually observable through instantaneous gel formation.

In yet another embodiment, it is preferable to carry out the esterification at a temperature below 50° C. and more preferably at a temperature of 37° C. The reaction is preferably carried out for about 15-30 mins.

In one aspect of the present invention, the activated cationized gelatin is reacted with targeting moiety or ligand of interest. Preferably, the ligand comprises of antibody. More preferably, IgG antibody is used as the targeting ligand and more preferably, EGFR targeting cetuximab is used as the targeting ligand.

In one embodiment, it is preferable to utilize the targeting ligand such as antibody, on an “as-is” basis, when there are no excipients containing free amine groups in the antibody.

In another embodiment, when free amines such as glycine are present as excipient, the antibody is purified and used for conjugation purposes.

In yet another embodiment, cetuximab (Erbitux) was purified using antibody capture chromatography (Protein A) and concentrated. In yet another embodiment, desalting of the antibody using methods such as but not limited to tangential flow filtration is carried out.

In yet another embodiment, it is preferable to have antibody concentration of more than 1 mg/ml, more preferably above 2 mg/ml and even more preferably above 5 mg/ml. In a preferred embodiment, the antibody concentration is maintained above 5 mg/ml.

In yet another embodiment, it is also preferable to maintain the pH of the antibody at basic conditions for the reaction to take place. It is preferable that the pH of the antibody solution is less than 11 prior to the addition of the activated cationized gelatin. It is more preferable that the pH is below 10 and even more preferable to have pH below 9.

It is observed that the addition of activated cationized gelatin to the targeting moiety reduces the pH drastically. Thus, it is preferable to maintain the pH above 6 through step-wise addition of activated cationized gelatin and simultaneous addition of a base such as but not limited to alkali metal hydroxide or sodium carbonate or sodium bicarbonate.

In yet another embodiment, upon conjugation, it is preferred to remove the excess cationized gelatin present in the reaction mixture. Excess cationized gelatin may interfere during the biomolecule coupling procedure, such as coupling to siRNA.

In yet another embodiment, after reaction completion, the purification is typically carried out either through precipitation of excess cationized gelatin, removal of gelatin through capture chromatography or by Antibody capture chromatography.

In yet another embodiment, antibody capture chromatography is preferred as all reaction components other than modified antibody or free antibody is removed in the flow-through and wash. The antibody or the conjugate is eluted using suitable buffer.

In yet another embodiment, prior to loading of the reaction mixture onto chromatography resin, it is preferable to dilute the reaction mixture using suitable buffers to a concentration lower than 20 mg/ml of cationized gelatin, more preferably lower than 10 mg/ml and even more preferably lower than 5 mg/ml.

In yet another embodiment, optimal mole ratio of cationized gelatin to antibody is determined. Lower mole ratio of cationized gelatin to antibody (mole ratio less than 5) yields improper conjugation while higher mole ratio, typically more than 50, causes issues during purification.

In a preferred embodiment, the reactions are performed using cationized gelatin and antibody in a molar ratio greater than 1:5 and lesser than 1:100. It is preferred to have a mole ratio between 1:20 and 1:80 and even more preferably between 1:20 and 1:50.

It is noteworthy to mention that all the antibody in the solution is modified or only a fraction of antibody in the solution is modified with gelatin to form antibody-gelatin conjugate depending upon the concentration of the molecules and the reaction parameters. However, it is preferred to have maximum conjugation with appropriate reaction conditions.

In yet another embodiment, the conjugation of the antibody to cationized gelatin is carried by activating the carboxyl residues of the antibody or modifying the antibody at specific sites for thiol reactivity. In a preferred embodiment, the antibody is subjected to coupling with 1-Ethyl-3-(3-dimethylaminopropyl) carbodiimide (EDC)/N-hydroxysuccinimide (NHS) at antibody concentration of 5 mg/ml or above. After 30 mins, the activated antibody is added to the cationized gelatin pre-dissolved at pH range of 7-11, preferably between 8-10 and more preferably at pH 9. The reaction is allowed for 0.5-2 hours, diluted and optionally purified using antibody capture chromatography.

In yet another embodiment, the antibody-macromolecule conjugate was reduced and analyzed for band shift using SDS gel electrophoresis. Several bands of conjugates (reduced) suggest various number of macromolecule linker attached to the antibody (FIG. 5).

In yet another embodiment, non-reduced SDS gel electrophoresis is used for analysis of intact band shift (FIG. 6).

In yet another embodiment, reverse phase HPLC (RP-HPLC) profile of antibody and antibody-macromolecule conjugate is performed and change in the retention time indicates the formation of the complex. The conversion % is estimated using area under the curve of antibody standard (FIG. 7).

In one aspect of the present invention, the coupling of siRNA to macromolecule-antibody conjugate is carried using two methods namely (i) chemical conjugation of ss-siRNA with macromolecule-antibody conjugate (FIG. 2) or (ii) physical entrapment of siRNA or ss-siRNA with macromolecule-antibody conjugate (FIG. 3).

In a preferred embodiment, siRNA is attached to the construct by converting the amines present in gelatin to thiol reactive maleimide functional group and thio-siRNA (ss-siRNA) covalently attached within the construct through thio-ether bond to form cAMB. Preferably, in the case of chemical conjugation, thiol present in the siRNA is linked to the amine residues present in antibody-gelatin conjugate.

In another embodiment, the antibody-gelatin conjugate is buffer-exchanged with phosphate buffer, concentrated and finally resuspended in Phosphate Buffer, pH 7 to achieve appropriate concentration.

In yet another embodiment, the concentration of the solution is preferably above 1 mg/ml, more preferably above 5 mg/ml and even more preferably above 15 mg/ml.

In yet another embodiment, m-maleimidobenzoyl-N-hydroxysulfosuccinimide ester (sulfo-MBS) is added to the antibody-macromolecule complex and the reaction is allowed to proceed for 15 mins to 2 hours at room temperature.

In yet another embodiment, excess sulfo-MBS is removed completely or partially through precipitation or buffer exchange or combination of both.

In yet another embodiment, the maleimide modified conjugate is resuspended in phosphate buffer at concentration above 1 mg/ml. Preferably, the concentration is maintained above 5 mg/ml, more specifically above 10 mg/ml and even more specifically at a concentration of about 15 mg/ml (FIG. 8).

In yet another embodiment, ss-siRNA in RNAse free water is added to the maleimide modified conjugate for thio-ether linkage of siRNA and antibody-gelatin complex to form cAMB. It is preferable to reduce the ss-siRNA prior to addition.

In yet another embodiment, cAMB is dual stained with ethidium bromide and Coomassie blue to observe the co-localization of siRNA and Protein (FIG. 9).

In yet another embodiment, the release of siRNA in the presence of cytoplasmic lysate was analysed through gel retardation assay. This assay proves that when the compound translocates to cytosplasm of the cells, siRNA release occurs. (FIG. 10).

In yet another preferred embodiment, siRNA (siRNA sequence: sense strand: 5′-GUUGGAGCUUGUGGCGUAGUU-3′ and antisense strand: 5′-CUACGCCACAAGCUCCAACUU-3′) directed against KRas G12C mutation is utilized.

In a preferred embodiment, a cAMB conjugate, comprising of an antibody, a macromolecule linker and siRNA is utilized for delivery of siRNA, gene knockdown and sensitization of cells towards a small molecule—Tyrosine Kinase inhibitor (TKI).

In a further preferred embodiment of the invention, the cAMB enables substantially lower amount of TKI required for sensitization of cells post oncogene knockdown wherein, a 10-fold lower concentration of the TKI reduces the viability of the cells extensively relative to the transient knockdown through naked siRNA and drug treatment.

Alternatively, in another aspect of the present invention, the siRNA is physically entrapped to the antibody-gelatin conjugate.

In one embodiment, the entrapment occurs through differential charge existing between siRNA which is negative and the antibody-gelatin conjugate which is positive. The charge is pH dependent and it is understood that there exists a pH for antibody-gelatin conjugate solution above which, the innate charge of the molecule becomes negative (FIG. 4b).

In another embodiment, it is preferred that the pH of the antibody-gelatin conjugate solution is below pH 10, more preferably below pH 9 and even more preferably below pH 8 for effective siRNA entrapment (FIG. 22).

In yet another embodiment, the concentration of the antibody-gelatin conjugate also plays an important role for the entrapment of siRNA to the antibody-gelatin conjugate. It is preferred that concentration of the antibody-gelatin conjugate be above 1 mg/ml for effective siRNA entrapment (FIG. 23).

In a preferred embodiment, the proof of concept is directed towards the delivery of siRNA to cells for specific gene knockdown.

In another embodiment, the biomolecule-antibody conjugate is designed for delivery of various siRNA specific to the mRNA sequence of interest.

In one of the embodiments of the invention, Immunoglobulin G (IgG) has been used as the targeting moiety to generate a global model.

In a preferred embodiment, the cAMB compounds or derivatives of the present invention is selected from Table 1.

TABLE 1
List of compounds

| Compound # | Formula |
|---|---|
| iv.a | IgG-cGelA225 |
| iv.b | IgG-cGelA110 |
| iv.c | Ctb-cGelA225 |
| iv.d | Ctb-cGelA110 |
| v.a | IgG-cGelA225-siRNA(thiol) |
| v.b | IgG-cGelA110-siRNA(thiol) |
| v.c | Ctb-cGelA225-siRNA(thiol) |
| v.d | Ctb-cGelA110-siRNA(thiol) |
| vi.a | IgG-cGelA225-siRNA |
| vi.b | IgG-cGelA110-siRNA |
| vi.c | Ctb-cGelA225-siRNA |
| vi.d | Ctb-cGelA110-siRNA |
| vii.a | IgG(A)-cGelA225 |
| vii.b | IgG(A)-cGelA110 |
| vii.c | Ctb(A)-cGelA225 |
| vii.d | Ctb(A)-cGelA110 |
| viii.a | IgG(A)-cGelA225-siRNA(thiol) |
| viii.b | IgG(A)-cGelA110-siRNA(thiol) |
| viii.c | Ctb(A)-cGelA225-siRNA(thiol) |
| viii.d | Ctb(A)-cGelA110-siRNA(thiol) |
| ix.a | IgG(A)-cGelA225-siRNA |
| ix.b | IgG(A)-cGelA110-siRNA |
| ix.c | Ctb(A)-cGelA225-siRNA |
| ix.d | Ctb(A)-cGelA110-siRNA |

The Antibody-Macromolecule-Biomolecule conjugate is for delivering a payload of biological molecule which is highly prone to degradation as well as molecules which are required to enter cytoplasm or nucleus of the cells for therapeutic effect.

The conjugate is contemplated to provide a scalable and homogeneous biological molecule for example siRNA delivery system which can either choose ‘off-the-shelf’ antibody or purified antibody to provide the conjugate delivery system.

The conjugate can be used to provide stable and targeted delivery of biological molecule for example siRNA to cells of interest and to translocate the delivered active biomolecule to various cellular compartments such as but not limited to cytoplasm, nucleus etc depending on the type of biomolecule. In the case of siRNA delivery, it is preferable for cytoplasmic translocation inside the cells upon endocytosis for gene knockdown.

In accordance with the disclosure, the macromolecule linker binds to biological molecule to the macromolecule-biomarker targeting moiety complex. The macromolecule linker serves the purpose of translocation of the biomolecule payload to other regions of the cell such as but not limited to cytoplasm and nucleus through endosomal escape mediated by the linker's potential for charge reversal and/or proton sponge effect. Also, the linker aides in protecting biological molecule like siRNA from degradation.

The present disclosure provides conjugate construct and the route of synthesis can allow for modification with a desired biomarker targeting moiety and biological molecule as per the patient requirement for clinical as well as diagnostic application(s).

### EXAMPLES

The disclosure will now be illustrated with following non-limiting working examples, which is intended to illustrate the working of disclosure and not intended to take restrictively to imply any limitations on the scope of the present disclosure. It is emphasized herein that antibody used in the examples are both IgG and Cetuximab. IgG is used to impart generality to the system whereas Cetuximab is used for specificity to EGFR present in reported NSCLC cell line.

### Example 1: Preparation of Cationized Gelatin

1 gm of type A gelatin bloom 110 (cGelA110) was dissolved in 40 ml of de-ionized water at 37° C. 250 mg of 1-Ethyl-3-(3-dimethylaminopropyl) carbodiimide (EDC) and 200 mg of N-Hydroxysuccinimide ester (NHS) was dissolved in 0.1M MES buffer (pH 2.5). The EDC/NHS solution was added to the pre-dissolved gelatin solution. Subsequently, 0.5 ml of 5N HCl was added to the above reaction mixture to reduce the pH of the solution. Immediately, 0.08 ml of 15M Ethylenediamine (EDA) was added to the above reaction mixture resulting in a final pH of approximately 5. The reaction was then allowed to proceed overnight at 37° C. under constant stirring. Subsequently, 3 volumes of acetone was added to the reaction mixture and the Cationized gelatin type A was precipitated. The solution was then centrifuged and washed with acetone thrice to remove excess EDA. Residual acetone present in the pellet was then removed under low pressure. The solution was resuspended in DI-Water and the cationization of the gelatin was checked by measuring the zeta-potential. Zeta potential increases from approximately +6 mV to +20V.

Same process was applied for cationization of type A gelatin bloom 225. Zeta potential increases from approximately +8 mV to approximately +25 mV.

### Example 2: Synthesis of N-Hydroxysuccinimide Ester of Cationized Gelatin A 110/Cationized Gelatin A 225 (cGelA110/cGelA225)

The cationized gelatin obtained from the above process was dissolved in 0.1M MES buffer (pH 2.5) at a concentration of 60 mg/ml. The pH of the cationized gelatin solution was then reduced to approximately 2.5 with 5N HCl. To 10 ml of this solution, 10 ml of a solution containing 300 mg of EDC and 200 mg of NHS pre-dissolved in 0.1M MES buffer (pH 2.5) was added. The pH of the solution was reduced to approximately to 2.5 with 5N HCl. The reaction was allowed to proceed for 15-30 mins at 37° C. This activated solution was taken forward for conjugating with antibody without any further processing.

### Example 3: Synthesis of IgG-Activated Cationized Gelatin A 110 Conjugate (IgG-cGelA110)

IgG available commercially contain salts and amino acids as excipients that can interfere with the chemical conjugation. Therefore, Protein A affinity chromatography was performed to remove the excipients and obtain pure IgG. Protein A resin was equilibrated with phosphate buffer solution (PBS, pH 7.5). After equilibration, the antibody was diluted to 10 mg/ml in equilibration buffer and loaded onto the resin at 100 cm/hr. The column was washed with equilibration buffer and the antibody was eluted using 0.1M acetic acid. After elution, the pH of the eluate was adjusted to 7.5 using 5M NaOH. The concentration of the antibody after purification was analyzed using Bradford's protein estimation and absorbance measurement (A280). Purity of the protein was analyzed through SDS gel electrophoresis and RP-HPLC. The eluate, containing purified IgG, was then concentrated to 10 mg/ml (pH7). To 1 ml of 10 mg/ml IgG, 40 mg of activated cGelA110 was added. 10% sodium carbonate was added simultaneously along with the cGelA110 to the antibody. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 60% and the effective conversion of IgG was approximately 75% determined using RP-HPLC. Confirmation of conjugation was also carried out through reducing SDS PAGE.

### Example 4: Synthesis of IgG-Activated Cationized Gelatin A 225 Conjugate (IgG-cGelA225)

Protein A affinity chromatography was performed to remove the excipients and obtain pure IgG as described in example 3. To 1 ml of 10 mg/ml purified IgG, 65 mg of activated cGelA225 was added. 10% sodium carbonate was added simultaneously along with the cGelA225 to the antibody. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 62% and the effective conversion of IgG was approximately 80% determined using RP-HPLC. Confirmation of conjugate was also carried out through reducing SDS PAGE.

### Example 5: Synthesis of Cetuximab-Activated Cationized Gelatin A 110 Conjugate (Ctb-cGelA110)

Protein A affinity chromatography was performed to remove the excipients and obtain pure cetuximab as described in example 3. To 1 ml of 10 mg/ml purified cetuximab, 40 mg of activated cGelA110 was added. 10% sodium carbonate was added simultaneously along with the cGelA110 to the antibody. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 65% and the effective conversion of IgG was approximately 75% determined using RP-HPLC. Confirmation of conjugate was also carried out through reducing SDS PAGE.

### Example 6: Synthesis of Cetuximab-Activated Cationized Gelatin A 225 Conjugate (Ctb-cGelA225)

Protein A affinity chromatography was performed to remove the excipients and obtain pure cetuximab as described in example 3. To 1 ml of 10 mg/ml purified cetuximab, 65 mg of activated cGelA225 was added. 10% sodium carbonate was added simultaneously along with the cGelA225 to the antibody. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 75% and the effective conversion of IgG was approximately 80% determined using RP-HPLC. Confirmation of conjugate was also carried out through reducing SDS PAGE.

### Example 7: Synthesis of IgG-cGelA110-siRNA(thiol)

5 mg of purified IgG-cGelA110 obtained from example 3 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized IgG-cGelA110 at a mole ratio of 1:1 (siRNA to IgG-cGelA110) to form IgG-cGelA110-siRNA. The reaction was carried out for 2 hours and the amount of bound siRNA was determined through RP-HPLC and Densitometry analysis.

### Example 8: Synthesis of IgG-cGelA225-siRNA(thiol)

5 mg of purified IgG-cGelA225 obtained from example 4 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized IgG-cGelA225 at a mole ratio of 1:1 (siRNA to IgG-cGelA225) to form IgG-cGelA225-siRNA. The reaction was carried out for 2 hours and the amount of bound siRNA was determined through RP-HPLC and Densitometry analysis.

### Example 9: Synthesis of Ctb-cGelA110-siRNA(thiol)

5 mg of purified Ctb-cGelA110 obtained from example 5 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized Ctb-cGelA110 at a mole ratio of 1:1 (siRNA to Ctb-cGelA110) to form Ctb-cGelA110-siRNA. The reaction was carried out for 2 hours and the amount of bound siRNA was determined through RP-HPLC and Densitometry analysis.

### Example 10: Synthesis of Ctb-cGelA225-siRNA(thiol)

5 mg of purified Ctb-cGelA225 obtained from example 6 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized Ctb-cGelA225 at a mole ratio of 1:1 (siRNA to Ctb-cGelA225) to form Ctb-cGelA225-siRNA. The reaction was carried out for 2 hours and the amount of bound siRNA was determined through RP-HPLC and Densitometry analysis.

### Example 11: Synthesis of IgG-cGelA110-siRNA (Electrostatic Interaction)

5 mg of purified IgG-cGelA110 obtained from example 3 was concentrated to approximately 5 mg/ml in 10 mM PBS (pH 7.2). 50 uM siRNA was added to this concentrated conjugate at a mole ratio of 1:1 (siRNA to IgG-cGelA110) to form IgG-cGelA110-siRNA. The sample was incubated at 37° C. for 5 minutes. The amount of bound siRNA was determined through RP-HPLC and Densitometry analysis.

### Example 12: Synthesis of IgG-cGelA225-siRNA (Electrostatic Interaction)

5 mg of purified IgG-cGelA225 obtained from example 4 was concentrated to approximately 5 mg/ml in 10 mM PBS (pH 7.2). 50 uM siRNA was added to this concentrated conjugate at a mole ratio of 1:1 (siRNA to IgG-cGelA225) to form IgG-cGelA225-siRNA. The sample was incubated at 37° C. for 5 minutes. The amount of bound siRNA was determined through RP-HPLC and Densitometry analysis.

### Example 13: Synthesis of Ctb-cGelA110-siRNA (Electrostatic Interaction)

5 mg of purified Ctb-cGelA110 obtained from example 5 was concentrated to approximately 5 mg/ml in 10 mM PBS (pH 7.2). 50 uM siRNA was added to this concentrated conjugate at a mole ratio of 1:1 (siRNA to Ctb-cGelA110) to form Ctb-cGelA110-siRNA. The sample was incubated at 37° C. for 5 minutes. The amount of bound siRNA was determined through RP-HPLC and Densitometry analysis.

### Example 14: Synthesis of Ctb-cGelA225-siRNA (Electrostatic Interaction)

5 mg of purified Ctb-cGelA225 obtained from example 6 was concentrated to approximately 5 mg/ml in 10 mM PBS (pH 7.2). 50 uM siRNA was added to this concentrated conjugate at a mole ratio of 1:1 (siRNA to Ctb-cGelA225) to form Ctb-cGelA225-siRNA. The sample was incubated at 37° C. for 5 minutes. The amount of bound siRNA was determined through RP-HPLC and Densitometry analysis.

### Example 15: Synthesis of N-Hydroxysuccinimide Ester of IgG/CTB

Commercially available antibody was purified through Protein A affinity chromatography. The eluate containing purified Antibody was buffer exchanged to 0.1M MES buffer (pH 2.5) at a concentration of 10 mg/ml. To 10 ml of this solution, 100 mg of EDC (at 50 mg/ml concentration) and 70 mg of NHS (at 60 mg/ml concentration) pre-dissolved in 0.1M MES buffer (pH 2.5) was added. The pH of the solution was reduced to approximately to 2.5 with 5N HCL. The reaction was allowed to proceed for 15-30 mins at RT. This activated solution was taken forward for conjugating with cationised Gelatin without any further processing.

### Example 16: Synthesis of Activated IgG-Cationized Gelatin A 110 Conjugate (IgG(A)-cGelA110)

Cationized gelatin A 110 was precipitated with 3 volumes of acetone. The solution was then centrifuged and washed thrice with acetone. Residual acetone present in the pellet was then removed under low pressure. The solution was resuspended in 100 mM PBS (pH 7.2) at 30 mg/ml. To 1 ml of 30 mg/ml cGelA110, 7.5 mg of activated IgG was added. 10% sodium carbonate was added simultaneously along with activated antibody to cGelA110. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 55% and the effective conversion of IgG was approximately 75% determined using RP-HPLC. Confirmation of conjugation was also carried out through reducing SDS PAGE.

### Example 17: Synthesis of Activated IgG-Cationized Gelatin A 225 Conjugate (IgG(A)-cGelA225)

Cationized gelatin A 225 was precipitated with 3 volumes of acetone. The solution was then centrifuged and washed thrice with acetone. Residual acetone present in the pellet was then removed under low pressure. The solution was resuspended in 100 mM PBS (pH 7.2) at 30 mg/ml. To 1 ml of 30 mg/ml cGelA225, 7.5 mg of activated IgG was added. 10% sodium carbonate was added simultaneously along with activated antibody to cGelA225. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 60% and the effective conversion of IgG was approximately 80% determined using RP-HPLC. Confirmation of conjugation was also carried out through reducing SDS PAGE.

### Example 18: Synthesis of Activated Cetuximab-Cationized Gelatin A 110 Conjugate (Ctb(A)-cGelA110)

Cationized gelatin A 110 was precipitated with 3 volumes of acetone. The solution was then centrifuged and washed thrice with acetone. Residual acetone present in the pellet was then removed under low pressure. The solution was resuspended in 100 mM PBS (pH 7.2) at 30 mg/ml. To 1 ml of 30 mg/ml cGelA110, 7.5 mg of activated CTB was added. 10% sodium carbonate was added simultaneously along with activated antibody to cGelA110. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 60% and the effective conversion of CTB was approximately 80% determined using RP-HPLC. Confirmation of conjugation was also carried out through reducing SDS PAGE.

### Example 19: Synthesis of Cetuximab-Activated Cationized Gelatin A 225 Conjugate (Ctb-cGelA225)

Cationized gelatin A 225 was precipitated with 3 volumes of acetone. The solution was then centrifuged and washed thrice with acetone. Residual acetone present in the pellet was then removed under low pressure. The solution was resuspended in 100 mM PBS (pH 7.2) at 30 mg/ml. To 1 ml of 30 mg/ml cGelA225, 7.5 mg of activated CTB was added. 10% sodium carbonate was added simultaneously along with activated antibody to cGelA225. The reaction was carried out at RT for 1 hr. Subsequently, the reaction mixture was clarified to remove particulates and was diluted with a buffer comprising of 10 mM PBS, 10 mM NaCl and 0.1 mM EDTA (pH 7). The diluted reaction mixture was then purified using protein A chromatography. The yield of purified conjugate was approximately 50% and the effective conversion of CTB was approximately 75% determined using RP-HPLC. Confirmation of conjugation was also carried out through reducing SDS PAGE.

### Example 20: Synthesis of IgG(A)-cGelA110-siRNA(thiol)

5 mg of purified IgG(A)-cGelA110 obtained from example 3 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized IgG(A)-cGelA110 at a mole ratio of 1:1 (siRNA to IgG(A)-cGelA110) to form IgG(A)-cGelA110-siRNA. The reaction was carried out for 2 hours and the amount of bound siRNA was determined through RP-HPLC and Densitometry analysis.

### Example 21: Synthesis of IgG(A)-cGelA225-siRNA(thiol

5 mg of purified IgG(A)-cGelA225 obtained from example 4 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized IgG(A)-cGelA225 at a mole ratio of 1:1 (siRNA to IgG(A)-cGelA225) to form IgG(A)-cGelA225-siRNA. The reaction was carried out for 2 hours and the amount of bound siRNA was determined through RP-HPLC and Densitometry analysis.

### Example 22: Synthesis of Ctb(A)-cGelA110-siRNA(thiol)

5 mg of purified Ctb(A)-cGelA110 obtained from example 5 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized Ctb(A)-cGelA110 at a mole ratio of 1:1 (siRNA to Ctb(A)-cGelA110) to form Ctb(A)-cGelA110-siRNA. The reaction was carried out for 2 hours and the amount of bound siRNA was determined through RP-HPLC and Densitometry analysis.

### Example 23: Synthesis of Ctb(A)-cGelA225-siRNA(thiol)

5 mg of purified Ctb(A)-cGelA225 obtained from example 6 was concentrated to 15 mg/ml in 10 mM PBS (pH 7.2). To 0.1 ml of the solution, m-maleimidobenzoyl-N-hydroxysulfosuccinimide ester (sulfo-MBS) was added to a final concentration of 1 mM sulfo-MBS. The reaction was allowed for 30 mins at RT and excess sulfo-MBS was removed through desalting. Subsequently, thio-siRNA (SS-siRNA) was added to maleimide functionalized Ctb(A)-cGelA225 at a mole ratio of 1:1 (siRNA to Ctb(A)-cGelA225) to form Ctb(A)-cGelA225-siRNA. The reaction was carried out for 2 hours and the amount of bound siRNA was determined through RP-HPLC and Densitometry analysis.

### Example 24: Synthesis of IgG(A)-cGelA110-siRNA (Electrostatic Interaction)

5 mg of purified IgG(A)-cGelA110 obtained from example 3 was concentrated to approximately 5 mg/ml in 10 mM PBS (pH 7.2). 50 uM siRNA was added to this concentrated conjugate at a mole ratio of 1:1 (siRNA to IgG(A)-cGelA110) to form IgG(A)-cGelA110-siRNA. The sample was incubated at 37° C. for 5 minutes. The amount of bound siRNA was determined through RP-HPLC and Densitometry analysis.

### Example 25: Synthesis of IgG(A)-cGelA225-siRNA (Electrostatic Interaction)

5 mg of purified IgG(A)-cGelA225 obtained from example 4 was concentrated to approximately 5 mg/ml in 10 mM PBS (pH 7.2). 50 uM siRNA was added to this concentrated conjugate at a mole ratio of 1:1 (siRNA to IgG(A)-cGelA225) to form IgG(A)-cGelA225-siRNA. The sample was incubated at 37° C. for 5 minutes. The amount of bound siRNA was determined through RP-HPLC and Densitometry analysis.

### Example 26: Synthesis of Ctb(A)-cGelA110-siRNA (Electrostatic Interaction)

5 mg of purified Ctb(A)-cGelA110 obtained from example 5 was concentrated to approximately 5 mg/ml in 10 mM PBS (pH 7.2). 50 uM siRNA was added to this concentrated conjugate at a mole ratio of 1:1 (siRNA to Ctb(A)-cGelA110) to form Ctb(A)-cGelA110-siRNA. The sample was incubated at 37° C. for 5 minutes. The amount of bound siRNA was determined through RP-HPLC and Densitometry analysis.

### Example 27: Synthesis of Ctb(A)-cGelA225-siRNA (Electrostatic Interaction)

5 mg of purified Ctb(A)-cGelA225 obtained from example 6 was concentrated to approximately 5 mg/ml in 10 mM PBS (pH 7.2). 50 uM siRNA was added to this concentrated conjugate at a mole ratio of 1:1 (siRNA to Ctb(A)-cGelA225) to form Ctb(A)-cGelA225-siRNA. The sample was incubated at 37° C. for 5 minutes. The amount of bound siRNA was determined through RP-HPLC and Densitometry analysis.

### Example 28: Protein Down-Regulation

In-vitro Western Blot analysis comprising of H23 NSCLC cells harboring KRas G12C mutation were treated with compound [v.c]—“Cetuximab-CGelA225-siRNAKras”, with appropriate controls. Western blot analysis revealed down-regulation of RAS downstream pathway protein and PI3K downstream pathway protein (FIG. 11).

### Example 29: Receptor Binding Study

Fluorescent microscopy analysis of H23 cells treated with various conjugates were performed to observe binding properties of the cAMB to H23 cell surface EGFR. It was observed that cetuximab, cetuximab-macromolecule conjugates and cetuximab-macromolecule-siRNA conjugates had specific affinity towards EGFR whereas IgG and IgG-macromolecule conjugate bound to EGFR non-specifically (FIG. 12-FIG. 17).

### Example 30: Receptor Binding Study

Surface plasmon resonance (SPR) analysis was carried out for various cAMB molecules vis-à-vis “Cetuximab-CGelA225”, “Cetuximab-CGelA225-siRNAKras”, “IgG-CGelA225”, “IgG-CGelA225-siRNAKras”, Cetuximab and IgG to determine binding affinity and binding kinetics against His tagged EGFR. The results revealed similar affinity and kinetics for cAMB comprising of cetuximab and standalone Cetuximab antibody. IgG and cAMB comprising of IgG antibody did not show any binding in the SPR analysis.

### Example 31: Cytotoxicity Study

In-vitro cytotoxicity assay analysis comprising of H23 NSCLC cells harboring KRas G12C mutation were treated with compound [v.c]—“Cetuximab-CGelA225-siRNAKras” and TKI—Gefitinib with appropriate controls. MTT cytotoxicity analysis revealed synergistic effect of oncogene knockdown and tyrosine kinase inhibition causes apoptosis of cells (FIG. 18-FIG. 21).

From the foregoing, it will be appreciated that, although specific embodiments of the invention have been described herein merely for purposes of illustration, various modifications may be made without deviating from the spirit and scope of the invention and should not be construed so as to limit the scope of the invention or the appended claims in any way.

1-39. (canceled)

40. A conjugate comprising entities as per Formula (1):
Aₙ-Lₓ-B_{y} Formula 1
wherein,
A is a biomarker targeting moiety selected from antibody, antibody fragment or peptide;
L is a macromolecule linker selected from one or more of protein, peptide, polypeptide or a polymer comprising at least one carboxyl group;
B is a biological molecule selected from one or more of nucleic acid(s) selected from siRNA, shRNA, or DNA; or a peptide;
n is 1;
x is 1-10; and
y is 0-100;
and wherein A is linked to L through amide bond, disulfide bond or thio-ether bond; and
B is attached to L through a thio-ether bond, a disulfide bond, or attached by electrostatic force of interaction, or physical entrapment or combination thereof.

41. The conjugate as claimed in claim 40 of Formula (1): Aₙ-Lₓ-B_{y}, wherein n is 1, x is 1-10 and y is 0.

42. The conjugate as claimed in claim 40 of Formula (1): Aₙ-Lₓ-B_{y}, wherein n is 1, x is 1-10 and y is ≥1.

43. The conjugate as claimed in claim 40, wherein the macromolecule linker is a cationic molecule, or modified to a cationic molecule.

44. The conjugate as claimed in claim 40, wherein the macromolecule linker is selected from the group consisting of gelatin, cationic gelatin, collagen, chitosan, dextran, dextrin, polyethyleneimine (PEI), Poly(L-Lysine), polyglutamic acid, analogue thereof, or derivative thereof.

45. The conjugate as claimed in claim 44, wherein the macromolecule linker is a gelatin or a cationic gelatin.

46. The conjugate as claimed in claim 40, wherein the biological molecule is siRNA.

47. The conjugate as claimed in claim 40, wherein the conjugate is further labeled with fluorescent dye or radioactive label.

48. A process for preparing a conjugate comprising entities as per Formula (1):
Aₙ-Lₓ-B_{y} Formula 1
wherein,
A is a biomarker targeting moiety selected from antibody, antibody fragment or peptide;
L is a macromolecule linker selected from one or more of protein, peptide, polypeptide, or a polymer comprising at least one carboxyl group;
B is a biological molecule selected from one or more of nucleic acid(s) selected from siRNA, shRNA, or DNA; or a peptide;
n is 1;
x is 1-10;
y is 0-100;
wherein the process comprises:
linking A to L chemically through amide bond, disulfide bond or thio-ether bond; and
when y is ≥1, attaching B to L chemically through a thio-ether bond, a disulfide bond; or by electrostatic force of interaction, or physical entrapment, or combination thereof.

49. The process as claimed in claim 48, wherein the linking of A to L is carried out by reacting amine present in A with amine reactive ester of L; or reacting amine reactive ester of A with amine of L; or reacting the thiol of cystine present in A with thiol of L.

50. The process as claimed in claim 48, wherein the macromolecule linker is cationic molecule, or modified to cationic molecule.

51. The process as claimed in claim 48, wherein B is attached to L by modifying the macromolecule linker to introduce at least one functional group selected from thiol (—SH), or amine (—NH₂).

52. The process as claimed in claim 51, wherein the amine group is introduced in the macromolecule through compound selected from the group consisting of ethylenediamine, spermidine, spermine, PEI, poly-lysine, arginine or combination thereof, or thiol reactivity is introduced through thiol-containing moieties selected from cystine, and/or thiol reactive moieties selected from gold residue or silver residue.

53. The process as claimed in claim 49, wherein the reactive ester is introduced through imide functional group of N-hydroxy imide ester, wherein the imide functional group is selected from succinimide or phthalimide; or reactive ester is introduced through hydroxy-benzotriazole ester or its derivatives.

54. The process as claimed in claim 48, wherein the macromolecule linker is selected from the group consisting of gelatin, cationic gelatin collagen, chitosan, dextran, dextrin, polyethyleneimine (PEI), Poly(L-Lysine), polyglutamic acid, cationic polypeptide, analogue thereof, or derivative thereof.

55. The process as claimed in claim 48, wherein the macromolecule linker is gelatin or cationic gelatin, modified by one or more of synthetic modification selected from cleavage, chemical modification of carboxyl groups to amine groups; or physical modification

56. The process as claimed in claim 55, wherein the cationic gelatin is modified using N-(3-dimethylaminopropyl)-N′-ethylcarbodiimide and N-hydroxysuccinimide at a pH below 5, preferably at a pH below 3 and more preferably at pH below 2.5 and at a concentration of cationic gelatin below 100 mg/ml, preferably at a concentration below 50 mg/ml and more preferably at a concentration of 30 mg/ml.

57. The process as claimed in claim 48, wherein the concentration of biomarker targeting moiety is more than 1 mg/ml, preferably above 2 mg/ml and more preferably above 5 mg/ml and pH of the biomarker targeting moiety prior to the addition of the macromolecule linker is less than 11, preferably the pH is below 10, and more preferably the pH is below 9 but above 6.

58. The process as claimed in claim 48, wherein the reactions are performed using macromolecule linker and biomarker targeting moiety in a molar ratio greater than 1:5 and lesser than 1:100, preferably at a molar ratio between 1:20 and 1:80; and more preferably between 1:20 and 1:50.

59. The process as claimed in claim 48, further comprises step(s) for purification of conjugate by removal of excess of macromolecule linker and/or biomarker targeting moiety.

60. The process as claimed in claim 48, wherein the biological molecule is siRNA.

61. Use of conjugate as claimed in claim 40 to release and deliver a payload of the biological molecule to a target site mediated by mechanism selected from enzymatic cleavage, pH change, salt concentrations or temperature, and achieve gene knockdown.

62. Use of conjugate as claimed in claim 40, wherein the conjugate is capable of delivering siRNA to adenocarcinoma of non-small cell lung cancer (NSCLC) harboring KRas mutation.


SEQUENCE LISTING (2 sequences)

SEQ ID NO: 1 - RNA, 21 nt, Artificial Sequence
  Directed against KRas G12C mutation
guuggagcuuguggcguaguu

SEQ ID NO: 2 - RNA, 21 nt, Artificial Sequence
  Directed against KRas G12C mutation
cuacgccacaagcuccaacuu